SEQUENCE LISTING (50 sequences)

SEQ ID NO: 1 - protein, 112 aa, synthetic construct
APTTTTKVDIAAFNPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 2 - protein, 112 aa, Methylorubrum extorquens
APTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 3 - protein, 21 aa, Methylorubrum extorquens
MAFRLSSAVLLAALVAAPAYA

SEQ ID NO: 4 - protein, 133 aa, Methylorubrum extorquens
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 5 - protein, 133 aa, synthetic construct
MAFRLSSAVLLAALVAAPAYAAPTTTTKVDIAAFDPDKDGWIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 6 - protein, 133 aa, synthetic construct
MAFRLSSAVLLAALVAAPAYAAPTTTTKVDIAAFDPDKDGTIDLKEALAAGSAAFDKLDPDKDGWLDAKELKGRVSEADLKKLDPDNDGTLDKKEYLAAVEAQFKAANPDNDGTIDARELASPAGSALVNLIR

SEQ ID NO: 7 - protein, 133 aa, synthetic construct
MAFRLSSAVLLAALVAAPAYAAPTTTTKVDIAAFDPDKDGTIDLKEALAAGSAAFDKLDPDKDGTLDAKELKGRVSEADLKKLDPDWDGTLDKKEYLAAVEAQFKAANPDNDGTIDARELASPAGSALVNLIR

SEQ ID NO: 8 - protein, 133 aa, synthetic construct
MAFRLSSAVLLAALVAAPAYAAPTTTTKVDIAAFDPDKDGTIDLKEALAAGSAAFDKLDPDKDGTLDAKELKGRVSEADLKKLDPDNDGWLDKKEYLAAVEAQFKAANPDNDGTIDARELASPAGSALVNLIR

SEQ ID NO: 9 - protein, 133 aa, synthetic construct
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKWEYLAAVEAQFKAANPDNDGTIDARELASPAGSALVNLIR

SEQ ID NO: 10 - protein, 133 aa, synthetic construct
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGWIDARELASPAGSALVNLIR

SEQ ID NO: 11 - protein, 112 aa, synthetic construct
APTTTTKVDIAAFDPDKDGWIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 12 - protein, 112 aa, synthetic construct
APTTTTKVDIAAFDPDKDGTIDLKEALAAGSAAFDKLDPDKDGWLDAKELKGRVSEADLKKLDPDNDGTLDKKEYLAAVEAQFKAANPDNDGTIDARELASPAGSALVNLIR

SEQ ID NO: 13 - protein, 112 aa, synthetic construct
APTTTTKVDIAAFDPDKDGTIDLKEALAAGSAAFDKLDPDKDGTLDAKELKGRVSEADLKKLDPDWDGTLDKKEYLAAVEAQFKAANPDNDGTIDARELASPAGSALVNLIR

SEQ ID NO: 14 - protein, 112 aa, synthetic construct
APTTTTKVDIAAFDPDKDGTIDLKEALAAGSAAFDKLDPDKDGTLDAKELKGRVSEADLKKLDPDNDGWLDKKEYLAAVEAQFKAANPDNDGTIDARELASPAGSALVNLIR

SEQ ID NO: 15 - protein, 112 aa, synthetic construct
APTTTTKVDIAAFDPDKDGTIDLKEALAAGSAAFDKLDPDKDGTLDAKELKGRVSEADLKKLDPDNDGTLDKWEYLAAVEAQFKAANPDNDGTIDARELASPAGSALVNLIR

SEQ ID NO: 16 - protein, 112 aa, synthetic construct
APTTTTKVDIAAFDPDKDGTIDLKEALAAGSAAFDKLDPDKDGTLDAKELKGRVSEADLKKLDPDNDGTLDKKEYLAAVEAQFKAANPDNDGWIDARELASPAGSALVNLIR

SEQ ID NO: 17 - protein, 133 aa, synthetic construct
MAFRLSSAVLLAALVAAPAYAAPTTTTKVDIAAFDPDKDGTIDLKEALAAGSAAFDKLDPDWDGTLDAKELKGRVSEADLKKLDPDNDGTLDKKEYLAAVEAQFKAANPDNDGTIDARELASPAGSALVNLIR

SEQ ID NO: 18 - protein, 133 aa, synthetic construct
MAFRLSSAVLLAALVAAPAYAAPTTTTKVDIAAFDPDKDGTIDLKEALAAGSAAFDKLDPDKDGTLDAWELKGRVSEADLKKLDPDNDGTLDKKEYLAAVEAQFKAANPDNDGTIDARELASPAGSALVNLIR

SEQ ID NO: 19 - protein, 112 aa, synthetic construct
APTTTTKVDIAAFDPDKDGTIDLKEALAAGSAAFDKLDPDWDGTLDAKELKGRVSEADLKKLDPDNDGTLDKKEYLAAVEAQFKAANPDNDGTIDARELASPAGSALVNLIR

SEQ ID NO: 20 - DNA, 23 bp, synthetic construct
  source 1..23: mol_type = other DNA
ggaccctgactgggacggaaccc

SEQ ID NO: 21 - DNA, 18 bp, synthetic construct
  source 1..18: mol_type = other DNA
agcttcttaaggtctgcc

SEQ ID NO: 22 - DNA, 28 bp, synthetic construct
  source 1..28: mol_type = other DNA
caatgacggatggctggacaagaaagag

SEQ ID NO: 23 - DNA, 18 bp, synthetic construct
  source 1..18: mol_type = other DNA
tcagggtccagcttctta

SEQ ID NO: 24 - DNA, 27 bp, synthetic construct
  source 1..27: mol_type = other DNA
cctggacaagtgggagtacttagcagc

SEQ ID NO: 25 - DNA, 18 bp, synthetic construct
  source 1..18: mol_type = other DNA
gttccgtcattgtcaggg

SEQ ID NO: 26 - DNA, 38 bp, synthetic construct
  source 1..38: mol_type = other DNA
caaagatgggtggatcgatctgaaagaggctttggcgg

SEQ ID NO: 27 - DNA, 18 bp, synthetic construct
  source 1..18: mol_type = other DNA
tccgggtcaaacgccgcg

SEQ ID NO: 28 - DNA, 28 bp, synthetic construct
  source 1..28: mol_type = other DNA
taaagatggttggctggacgccaaagag

SEQ ID NO: 29 - DNA, 18 bp, synthetic construct
  source 1..18: mol_type = other DNA
tccgggtccaacttgtcg

SEQ ID NO: 30 - DNA, 24 bp, synthetic construct
  source 1..24: mol_type = other DNA
caacgatggctggattgacgcccg

SEQ ID NO: 31 - DNA, 18 bp, synthetic construct
  source 1..18: mol_type = other DNA
tcagggttagcggcctta

SEQ ID NO: 32 - DNA, 20 bp, synthetic construct
  source 1..20: mol_type = other DNA
taatacgactcactataggg

SEQ ID NO: 33 - DNA, 19 bp, synthetic construct
  source 1..19: mol_type = other DNA
gctagttattgctcagcgg

SEQ ID NO: 34 - protein, 112 aa, synthetic construct
APTTTTKVDIAAFDPDKDGTIDLKEALAAGSAAFDKLDPDKDGTLDAWELKGRVSEADLKKLDPDNDGTLDKKEYLAAVEAQFKAANPDNDGTIDARELASPAGSALVNLIR

SEQ ID NO: 35 - protein, 133 aa, synthetic construct
  SITE 41: note = X is a sensitizer
MAFRLSSAVLLAALVAAPAYAAPTTTTKVDIAAFDPDKDGXIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 36 - protein, 133 aa, synthetic construct
  SITE 65: note = X is a sensitizer
MAFRLSSAVLLAALVAAPAYAAPTTTTKVDIAAFDPDKDGTIDLKEALAAGSAAFDKLDPDKDGXLDAKELKGRVSEADLKKLDPDNDGTLDKKEYLAAVEAQFKAANPDNDGTIDARELASPAGSALVNLIR

SEQ ID NO: 37 - protein, 133 aa, synthetic construct
  SITE 87: note = X is a sensitizer
MAFRLSSAVLLAALVAAPAYAAPTTTTKVDIAAFDPDKDGTIDLKEALAAGSAAFDKLDPDKDGTLDAKELKGRVSEADLKKLDPDXDGTLDKKEYLAAVEAQFKAANPDNDGTIDARELASPAGSALVNLIR

SEQ ID NO: 38 - protein, 133 aa, synthetic construct
  SITE 90: note = X is a sensitizer
MAFRLSSAVLLAALVAAPAYAAPTTTTKVDIAAFDPDKDGTIDLKEALAAGSAAFDKLDPDKDGTLDAKELKGRVSEADLKKLDPDNDGXLDKKEYLAAVEAQFKAANPDNDGTIDARELASPAGSALVNLIR

SEQ ID NO: 39 - protein, 133 aa, synthetic construct
  SITE 94: note = X is a sensitizer
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKXEYLAAVEAQFKAANPDNDGTIDARELASPAGSALVNLIR

SEQ ID NO: 40 - protein, 133 aa, synthetic construct
  SITE 114: note = X is a sensitizer
MAFRLSSAVLLAALVAAPAYAAPTTTTKVDIAAFDPDKDGTIDLKEALAAGSAAFDKLDPDKDGTLDAKELKGRVSEADLKKLDPDNDGTLDKKEYLAAVEAQFKAANPDNDGXIDARELASPAGSALVNLIR

SEQ ID NO: 41 - protein, 133 aa, synthetic construct
  SITE 62: note = X is a sensitizer
MAFRLSSAVLLAALVAAPAYAAPTTTTKVDIAAFDPDKDGTIDLKEALAAGSAAFDKLDPDXDGTLDAKELKGRVSEADLKKLDPDNDGTLDKKEYLAAVEAQFKAANPDNDGTIDARELASPAGSALVNLIR

SEQ ID NO: 42 - protein, 133 aa, synthetic construct
  SITE 69: note = X is a sensitizer
MAFRLSSAVLLAALVAAPAYAAPTTTTKVDIAAFDPDKDGTIDLKEALAAGSAAFDKLDPDKDGTLDAXELKGRVSEADLKKLDPDNDGTLDKKEYLAAVEAQFKAANPDNDGTIDARELASPAGSALVNLIR

SEQ ID NO: 43 - protein, 112 aa, synthetic construct
  SITE 20: note = X is a sensitizer
APTTTTKVDIAAFDPDKDGXIDLKEALAAGSAAFDKLDPDKDGTLDAKELKGRVSEADLKKLDPDNDGTLDKKEYLAAVEAQFKAANPDNDGTIDARELASPAGSALVNLIR

SEQ ID NO: 44 - protein, 112 aa, synthetic construct
  SITE 44: note = X is a sensitizer
APTTTTKVDIAAFDPDKDGTIDLKEALAAGSAAFDKLDPDKDGXLDAKELKGRVSEADLKKLDPDNDGTLDKKEYLAAVEAQFKAANPDNDGTIDARELASPAGSALVNLIR

SEQ ID NO: 45 - protein, 112 aa, synthetic construct
  SITE 66: note = X is a sensitizer
APTTTTKVDIAAFDPDKDGTIDLKEALAAGSAAFDKLDPDKDGTLDAKELKGRVSEADLKKLDPDXDGTLDKKEYLAAVEAQFKAANPDNDGTIDARELASPAGSALVNLIR

SEQ ID NO: 46 - protein, 112 aa, synthetic construct
  SITE 69: note = X is a sensitizer
APTTTTKVDIAAFDPDKDGTIDLKEALAAGSAAFDKLDPDKDGTLDAKELKGRVSEADLKKLDPDNDGXLDKKEYLAAVEAQFKAANPDNDGTIDARELASPAGSALVNLIR

SEQ ID NO: 47 - protein, 112 aa, synthetic construct
  SITE 73: note = X is a sensitizer
APTTTTKVDIAAFDPDKDGTIDLKEALAAGSAAFDKLDPDKDGTLDAKELKGRVSEADLKKLDPDNDGTLDKXEYLAAVEAQFKAANPDNDGTIDARELASPAGSALVNLIR

SEQ ID NO: 48 - protein, 112 aa, synthetic construct
  SITE 93: note = X is a sensitizer
APTTTTKVDIAAFDPDKDGTIDLKEALAAGSAAFDKLDPDKDGTLDAKELKGRVSEADLKKLDPDNDGTLDKKEYLAAVEAQFKAANPDNDGXIDARELASPAGSALVNLIR

SEQ ID NO: 49 - protein, 112 aa, synthetic construct
  SITE 41: note = X is a sensitizer
APTTTTKVDIAAFDPDKDGTIDLKEALAAGSAAFDKLDPDXDGTLDAKELKGRVSEADLKKLDPDNDGTLDKKEYLAAVEAQFKAANPDNDGTIDARELASPAGSALVNLIR

SEQ ID NO: 50 - protein, 112 aa, synthetic construct
  SITE 48: note = X is a sensitizer
APTTTTKVDIAAFDPDKDGTIDLKEALAAGSAAFDKLDPDKDGTLDAXELKGRVSEADLKKLDPDNDGTLDKKEYLAAVEAQFKAANPDNDGTIDARELASPAGSALVNLIR

(19) United States
(12) Patent Application Publication
(10) Pub. No.: US 2024/0301009 A1
(43) Pub. Date: Sep. 12, 2024

(54) A PROTEIN-BASED SENSOR FOR METALS IN ENVIRONMENTAL SAMPLES AND USES THEREOF

(71) Applicant: The Penn State Research Foundation, University Park, PA (US)

(72) Inventors: Joseph Alfred COTRUVO, Jr., University Park, PA (US); Emily Rowland FEATHERSTON, University Park, PA (US); Joseph A. MATTOCKS, University Park, PA (US)

(21) Appl. No.: 18/578,302

(22) PCT Filed: Jul. 19, 2022
(86) PCT No.: PCT/US2022/073911

§ 371 Date: Jan. 10, 2024

Related U.S. Application Data
(60) Provisional application No. 63/223,522, filed on Jul. 19, 2021.

(51) Int. Cl. C07K 14/195; G01N 33/18; G01N 33/24

(52) U.S. Cl. CPC C07K 14/195; G01N 33/1813; G01N 33/24; C07K 2319/20

(57) ABSTRACT

The present disclosure provides proteins that bind lanthanides and actinides. Disclosed are proteins based on lanmodulin (LanM) that comprised one or more sensitizers. Various residues in LanM may be substituted with a sensitizer, such as, for example, tryptophan. The proteins may be used to detect and quantify lanthanides and actinides. Also provided are kits and devices.

### CROSS REFERENCE TO RELATED APPLICATION

This application claims priority to U.S. Provisional Application No. 63/223,522, filed on Jul. 19, 2021, the disclosure of which is incorporated herein by reference.

### STATEMENT REGARDING FEDERALLY SPONSORED RESEARCH

This invention was made with government support under Grant No. CHE1945015 awarded by the National Science Foundation and DE-SC0021007 awarded by Department of Energy. The Government has certain rights in the invention.

### SEQUENCE LISTING

The instant application contains a Sequence Listing, which has been submitted electronically in XML format and is hereby incorporated by reference in its entirety. The XML copy, created Jul. 18, 2022, is named “074339_00231_ST26.xml” and is 54,214 bytes.

### BACKGROUND OF THE DISCLOSURE

The rare earth elements (REEs)—a family of elements comprising the 15 lanthanides, plus yttrium and scandium-possess similar physiochemical properties and play indispensable roles in the emerging green economy. With increasing technological dependence on these elements, however, the chemical, environmental, and political challenges associated with mining and processing REEs have been magnified. These complexities have driven interest in obtaining REEs more sustainably from low-grade but abundant non-traditional sources, such as coal byproducts, mine effluents [e.g., acid mine drainage (AMD)], and recycling from electronic waste (E-waste). The discovery that biology is able to selectively recognize and utilize the lighter lanthanides, especially La—Nd, offers new possibilities for efficient biotechnologies to meet these challenges. This promise has been accelerated with the recent identification of the first native, selective biological chelator for the lanthanides, lanmodulin (LanM). This small (12-kDa) protein undergoes a conformational change that is 10⁸-fold more selective for lanthanides (and especially the lighter REEs) over non-REEs tested to date. Lanmodulin is the first natural, selective macrochelator for f-elements—a protein that binds lanthanides with picomolar affinity at 3 EF-hands, motifs that instead bind calcium in most other proteins. The protein tolerates acidic conditions (pH<2) relevant to environmental REE streams and industrial processes, and it is able to quantitatively extract REEs from acidic coal and e-waste leachates with high purity, outperforming traditional chelators. From a chemical perspective, this selectivity for REEs is all the more remarkable because the protein utilizes EF hands, carboxylate-rich metal-binding motifs associated with Ca^{II} recognition in most of the hundreds of other characterized examples.

Biochemical characterization of Methylorubrum extorquens LanM suggested that binding of REEs to two of the protein's three metal-binding sites occurs cooperatively with a picomolar apparent K_{d}, inducing formation of two-thirds of the protein's helical content; a third site binds with similar but slightly weaker affinity, contributing the remaining third. The NMR structure of lanmodulin revealed the overall structure of the protein, the three REE-binding EF hands (EF1-3), and a hydrophobic core that may help stabilize the REE-bound state of the protein (FIG. 1), but it left many questions unanswered. Although the protein architecture is unusual for an EF-hand protein, the pairing of EF2 and EF3 suggested that they might account for the cooperative metal-binding phase. Alternatively, the unusual connection of EF1 and EF2 through a single, short α-helix made stabilization of this helix via cooperative metal binding to EF1 and EF2 also a plausible source of the cooperative phase (FIG. 2). Furthermore, the detailed structure of the sites—in particular, whether solvent molecules contributed to the coordination spheres of the metal ions—could not be determined from the NMR structure. Therefore, the means by which structural and kinetic properties of the metal sites contribute to LanM's unique REE affinity and selectivity still remain to be determined.

Several lanthanide(III) ions (Ln^{III}) display intrinsic luminescence with relatively long luminescence lifetimes, especially in the cases of Tb^{III} and Eu^{III}, and diagnostic emission spectra.

In addition to their utility in biology and as biochemical probes, sensitive methods for detection of REEs are essential both in the identification of potential waste streams for REE recovery and in monitoring of industrial REE processing operations. Inductively coupled plasma mass spectrometry (ICP-MS) is the gold standard but expensive and not portable, whereas portable instruments like x-ray fluorescence spectrometers are limited by interferences and low sensitivities. As recently comprehensively reviewed, luminescence-based methods are attractive alternatives, especially in the case of Tb(III), which is both readily sensitized and one of the rarest and most valuable critical REEs. Despite recent progress to decrease limits of detection (LODs) even at low pH and in AMD matrix, popular approaches utilizing small molecules or metal-organic frameworks (MOFs) still lack the necessary selectivity, requiring spiking of samples with Tb for detection. By contrast, biomolecular luminescence-based sensors for Tb(III) based on EF hands such as lanthanide-binding tags (LBTs) exhibit better selectivity but their affinities (K_{d}˜50 nM or higher at pH 7) are insufficient for applications below pH ˜6. As a step toward more sensitive detection of REEs, our laboratory recently reported a selective FRET-based sensor for REEs (LaMP1), taking advantage of LanM's large conformational response. LaMP1 facilitated discovery of key elements of lanthanide uptake machinery in bacteria, but its use is limited to near-neutral pH values, it cannot distinguish between REEs, and it exhibits a small but significant response to some non-REEs at high concentrations. Consequently, it is unsuitable for complex environmental samples.

### BRIEF SUMMARY OF THE DISCLOSURE

Described herein is the strategic insertion of one or more sensitizers (e.g., luminescence sensitizers, e.g., tryptophan residues), into lanmodulin, which enables definition of aspects of the protein's selective REE recognition, including metal site-specific thermodynamics, kinetics, and structure. These insights also suggest how the protein might be optimized further for biotechnological applications. Furthermore, the present disclosure shows that these Trp-LanM variants enable detection and quantification of Tb levels directly in AMD, a challenging matrix inaccessible to previously characterized luminescent sensors. Together, these data suggest that this technology could be extended for detection of other luminescent f-elements and that LanM might enable harvesting of REEs from AMD.

The present disclosure provides proteins that bind lanthanides and/or actinides. Also provided are devices and kits comprising a protein of the present disclosure. Also provided are methods of using the proteins and devices.

In an aspect, the present disclosure provides proteins that bind metals (e.g., lanthanides and/or actinides). At least one residue of the protein is replaced with a sensitizer or the protein is modified such that a sensitizer is attached to the protein (e.g., attached to an amino acid).

In an aspect, the present disclosure provides devices. The device comprises one or more proteins of the present disclosures.

In an aspect, the present disclosure provides kits. The kits may provide one or more proteins of the present disclosure and/or one or more devices of the present disclosure. The kit may include instructions for use of the proteins or devices.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

A method of using a protein and/or device of the present disclosure may be a method for binding one or more lanthanides and/or actinides in a sample. Binding may occur by contacting the sample with one or more proteins and/or devices of the present disclosure. The method may be performed on various types of samples. Examples of samples include, but are not limited to drinking water, wastewater, ground water, ash ponds, aqueous extract from contaminated soil, drainage (e.g., mine drainage, such as, for example, acidic mine drainage) or leachate (e.g., landfill leachate). In various other example, the sample is a solid sample. The method may applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

A method of the present disclosure may be a method of detecting and/or quantifying the amount of one or more lanthanides and/or actinides in a sample. The method may comprise contacting the sample with one or more proteins and/or device of the present disclosure. The contacted sample may then be exposed to light and the resulting emission of the exposed contacted sample. The resulting emission results may then be compared to a known standard curve for a specific lanthanide or actinide. The concentration may then be determined by that comparison. Known standard curves may be prepared based on the desire to detect and/or determine the quantity of any specific lanthanide or actinide. Methods of preparing standard curves are known in the art.

In an aspect, the present disclosure provides a method to screen LanM variants using sensitized terbium luminescence, for altered metal ion selectivity (for separation applications). For example, a method uses a LanM containing a sensitizer (e.g., tryptophan at position 87, 90, or 94, or the equivalents in EF hands 1, 2, and 4).

### BRIEF DESCRIPTION OF THE FIGURES

For a fuller understanding of the nature and objects of the disclosure, reference should be made to the following detailed description taken in conjunction with the accompanying figures.

FIG. 1. A) NMR solution structure of Y^{III}-bound LanM, highlighting EF hands 1-4, with Y^{III} ions shown as spheres. B) Pairing of EF2 (right) and EF3 (left), indicating sites of individual Trp substitutions in sticks, at EF3 positions N87, T90, and K94, and EF2 position T65. C) Individual Trp substitutions in EF4 at position T114 (left) and EF1 at position T41 (right) indicated by sticks.

FIG. 2. The last residue of EF1 (Glu46) and the first residue of EF2 (Asp59) share a short, common helix. Metal coordinating residues are shown as sticks, and the EF hands are shown in gray. Alpha helices of this length are often poised between order and disorder, suggesting the possibility, investigated here, that metal binding to one EF hand and concomitant loop stabilization could be communicated to the other EF hand.

FIG. 3. Preliminary stoichiometric LRET titrations of LanM proteins containing Trp substitutions at (A) N87W (B) T90W and (C) K94W. (D-F) Expanded 515-575 nm range to highlight the emission feature at ˜545 nm for each construct. Note that N87W and K94W exhibit increases in Trp fluorescence upon metal binding but minimal Tb^{III} emission, whereas T90W exhibits quenching of Trp emission along with efficient energy transfer to Tb^{III}. Experimental parameters: 280 nm excitation, 400-700 nm emission, 5 nm excitation and emission slit widths, 120 nm/min scan rate, 1 nm data interval, 250-395 nm excitation filter, 430-1100 nm emission filter.

FIG. 4. Stoichiometric LRET titrations of LanM proteins containing Trp substitutions at (A) T41W (B) T65W and (C) T114W. (D-F) Expanded 515-575 nm range to highlight the emission feature at ˜545 nm for each construct. Experimental parameters: 280 nm excitation, 400-700 nm emission, 5 nm excitation and emission slit widths, 120 nm/min scan rate, 1 nm data interval, 250-395 nm excitation filter, 430-1100 nm emission filter.

FIG. 5. Characterization of energy transfer in Trp-LanM upon Eu^{III} binding monitored by spectrofluorometry. Energy transfer from Trp to Eu^{III} caused quenching of the Trp emission, but no significant Eu^{III} emission features were observed. The failure to observe significant Trp-sensitized Eu^{III} emission under these conditions is consistent with other protein systems. Experimental parameters: 275 nm excitation, 400-700 nm emission, 250-395 nm excitation filter, 430-1100 nm emission filter.

FIG. 6. Stoichiometric titrations of 15 μM wild-type, T41W, T65W, N87W, T90W, K94W, and Ti 14W LanMs with TbCl₃, followed using CD spectroscopy. The disruption of the conformational response of T65W is particularly noteworthy.

FIG. 7. Determination of apparent K_{d} values for Tb^{III}-LanM complexes by CD spectroscopy, for 15 μM (A) wild-type (B) T41W (C) T90W (D) K94W and (E) T114W. [θ]_{222 nm} was monitored at various EDDS-buffered free Tb^{III} ion concentrations, and data were fitted to the Hill equation to determine K_{d,app} and n. The values from the fits are shown in Table 3. Mean±SD for each point from triplicate measurements are displayed.

FIG. 8. Using Tb^{III} LRET to probe metal binding order in LanM. A) Example LRET spectra from Tb-EDDS buffered titration of T41W LanM. B) Overlay of representative K94W and Ti 14W LRET curves after subtraction of solutions containing the same Tb^{III} and EDDS concentrations, but without protein. Data were analyzed by averaging the luminescence signal at 544-546 nm plotted against [Tb^{III}_{free}], and fitted to the Hill equation. C) Model for the order of Tb^{III} binding to LanM, as proposed based on comparison of CD and LRET data of wt and Trp-LanM variants.

FIG. 9. Representative titration curves from determination of LRET-based K_{d,app} values for 10 μM (A) T41W (B) T90W (C) K94W (D) Ti 14W LanM variants. Tb^{III} emission was monitored from 400-700 nm with at a fixed excitation at 295 nm, at various EDDS-buffered free Tb^{III} ion concentrations. Luminescence intensities at 544-546 nm were averaged and, to remove the contribution from Tb-EDDS luminescence, values from a control experiment in the absence of protein were subtracted. The resulting values were fitted to the Hill equation to determine K_{d,app} and n.

FIG. 10. Determination of the luminescence lifetime of Tb^{III} bound to EF1. A) T41W-LanM luminescence decay curves in 0, 25, 50, and 75% D₂O. The data from each curve (carried out in triplicate) were fitted to a single exponential decay. B) Linear dependence of the decay time constant at different mole fractions of D₂O. τ(D₂O) was determined from the y-intercept.

FIG. 11. Determination of the luminescence lifetime of Tb^{III} in the T90W variant. A) T90W-LanM luminescence decay curves in 0, 25, 50, and 75% D₂O. The data from each curve (carried out in triplicate) were fitted to a single exponential decay. B) Linear dependence of the decay time constant at different mole fractions of D₂O. τ(D₂O) was determined from the y-intercept.

FIG. 12. Determination of the luminescence lifetime of Tb^{III} in the K94W variant. A) K94W-LanM luminescence decay curves in 0, 25, 50, and 75% D₂O. The data from each curve (carried out in triplicate) were fitted to a single exponential decay. Because of the low luminescence intensity of this variant, these experiments were carried out at 30 μM protein. B) Linear dependence of the decay time constant at different mole fractions of D₂O. τ(D₂O) was determined from the y-intercept.

FIG. 13. Determination of Tb^{III} dissociation kinetics by stopped-flow spectrofluorometry. Experimental curves were fitted to a single (T41W) or double (T90W) exponential decay and rate constants were plotted (mean±SD, n=3) were plotted against EGTA concentration. The k_{off} values were determined from the y-intercepts of the linear fits.

FIG. 14. Stopped flow spectrofluorometry of T41W- and T90W-LanM. A) T41W stopped-flow traces including single exponential fits (black lines). B) T90W stopped-flow traces including double exponential fits (black lines). C) Representative residual from the single exponential fitting of a trace from the T90W 5 mM EGTA data set. D) Representative residual from the double exponential fitting of the same trace, showing the necessity of the double exponential fit. There was not a full decrease in fluorescence over the measured time periods, likely due to some Trp fluorescence reaching the detector even with the 450 nm LP filter.

FIG. 15. Calibration curves for Trp-LanM-sensitized Tb luminescence at the major peak (F544-546 nm) versus [Tb^{III}] (ppb) with 1 μM (A) T41W and (B) T90W, from which LODs were determined. C) Similar curves for T90W at 10 μM, pH 3 and 4). The similar slopes under these conditions (18.8 at pH 3, 19.8 at pH 4) suggests similar saturation of the protein at both pH values, which bracket the pH of the AMD sample analyzed below.

FIG. 16. T90W-LanM quantifies Tb in AMD. A) Luminescence spectra of AMD with and without 10 μM T90W-LanM. B) Same as A, but also including spectra upon addition of 5-25 ppb Tb for generation of a calibration curve. C) Standard curve obtained from the data in (B), with the equation of the regression line used to quantify Tb in the AMD sample.

FIG. 17. A schematic of Trp-LanM binding Tb, a schematic of the binding of lanthanides to LanM, and sample data of environmental sensing of Tb at 3 ppb using Trp-LanM.

FIG. 18. Based on Trp-LanM results that showed that EF1 is the most labile lanthanide binding site in the protein, this EF hand was inactivated by mutation of its first residue, D35, to N. The LanM(D35N) variant was inspired by the presence of an N at the corresponding position in EF4 of the wt protein. This variant was tested for REE-binding stoichiometry and affinity. All experiments performed with 20 μM protein. A) Tyrosine fluorescence quenching experiments performed at pH 5.0 in 100 mM KCl, 30 mM MOPS, showing ˜2 equivalents of La(III) binding. B) Affinity for La(III) was tested by circular dichroism spectroscopy in 10 mM HEPES, 100 mM NaCl, pH 7 using EGTA as a competitive chelator. Results demonstrate comparable K_{d} for La(III)-LanM(D35N) (2.7 μM with Hill coefficient n=3.2) as with wt-LanM; therefore, these data suggest that we have disabled the weaker EF1 without affecting the metal binding characteristics of the rest of the protein (EF2/EF3). The sequence for LanM(D35N) is APTTTTKVDIAAFNPDKDGTIDLKEALAAGSAAFDKLDPDKDG TLDAKELKGRVSEADLKKLDPDNDGTLDKKEYLAAVEAQFKAANPDNDGTIDAREL ASPAGSALVNLIR (SEQ ID NO:1).

FIG. 19. K62W LRET curve after subtraction of solutions containing the same Tb^{III} and EDDS concentrations but without protein. Data were analyzed by averaging the luminescence signal at 544-546 nm plotted against [Tb^{III}_{free}] and fitted to the Hill equation (values shown in Table 8).

FIG. 20. K69W LRET curve after subtraction of solutions containing the same Tb^{III} and EDDS concentrations but without protein. Data were analyzed by averaging the luminescence signal at 544-546 nm plotted against [Tb^{III}_{free}] and fitted to the Hill equation (values shown in Table 8).

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

All ranges provided herein include all values that fall within the ranges to the tenth decimal place, unless indicated otherwise.

Described herein is the strategic insertion of one or more sensitizers (e.g., luminescence sensitizers, e.g., tryptophan residues), into lanmodulin, which enables definition of aspects of the protein's selective REE recognition, including metal site-specific thermodynamics, kinetics, and structure. These insights also suggest how the protein might be optimized further for biotechnological applications. Furthermore, the present disclosure shows that these Trp-LanM variants enable detection and quantification of Tb levels directly in AMD, a challenging matrix inaccessible to previously characterized luminescent sensors. Together, these data suggest that this technology could be extended for detection of other luminescent f-elements and that LanM might enable harvesting of REEs from AMD.

The present disclosure provides proteins that bind lanthanides and/or actinides. Also provided are devices and kits comprising a protein of the present disclosure. Also provided are methods of using the proteins and devices.

In an aspect, the present disclosure provides proteins that bind metals (e.g., lanthanides and/or actinides). At least one residue of the protein is replaced with a sensitizer or the protein is modified such that a sensitizer is attached to the protein (e.g., attached to an amino acid). Other metal-binding proteins are disclosed in WO2020051274, which is incorporated herein by reference.

Wt LanM without the signal peptide has the following sequence: APTTTTKVDIAAFDPDKDGTIDLKEALAAGSAAFDKLDPDKDGTLDAKELKGRVSE ADLKKLDPDNDGTLDKKEYLAAVEAQFKAANPDNDGTIDARELASPAGSALVNLIR (SEQ ID NO:2). The signal peptide has the following sequence: MAFRLSSAVLLAALVA APAYA (SEQ ID NO:3). The full length lanmodulin (including the signal peptide is MAFRLSSAVLLAALVAAPAYAAPTTTTKVDIAAFDPDKDGTIDLKEALAAGSAAFD KLDPDKDGTLDAKELKGRVSEADLKKLDPDNDGTLDKKEYLAAVEAQFKAANPDN DGTIDARELASPAGSALVNLIR (SEQ ID NO:4).

A protein of the present disclosure may be of various lengths. For example, a protein of the present disclosure has 80 to 160 amino acid residues, including all integer amino acid values and ranges therebetween. For example, the protein has a molecular weight of 10 kDa to 14 kDa, including all 0.1 Da values and ranges therebetween (e.g., ˜12 kDa). A protein of the present disclosure comprises at least one segment where one or more lanthanides and/or actinides can bind. The segment may have the same sequence of LanM, where at least one amino acid residue is replaced with a sensitizer or the protein is modified such that a sensitizer is attached to the protein (e.g., attached to an amino acid). In various examples, the segment has at least 70% homology (e.g., at least 70%, at least 75%, at least 80%, at least 85%, at least 90%, or at least 95% homology) with the sequence of Methylorubrum extorquens AM1 LanM, which may be referred to as LanM. In various other examples, the protein is truncated. For example, the protein is truncated at the N-terminus via deletion of the first 10, 20, 30, or 40 residues of the full translated sequence. In various examples of truncated sequences, EF hands 2 and 3 remain, as well as the hydrophobic core of the protein.

Suitable LanM proteins include the wild type M. extorquens LanM protein, or homologs from other organism having at least two EF hand motifs, with at least one EF hand motifs having at least 3 carboxylate residues, and at least 2 of the EF hand motifs being separated by a space of 10-15 residues. Reference herein will be made generally to “lanmodulin,” “LanM” or “LanM protein” and should be understood to include the wild type and homologs described herein. “LanM” can include full proteins having one or more LanM units or portions thereof comprising the one or more LanM units. LanM units include at least two EF hand motifs, with at least one EF hand motifs having at least 3 carboxylate residues, and at least 2 of the EF hand motifs being separated by a space of 10-15 residues. For ease of reference, discussion will be made with reference to lanmodulin, LanM or LanM protein and should be understood to include both the full proteins and portions of full proteins having the suitable LanM unit.

Various amino acid residues of the segment may be replaced with one or more sensitizers or the protein is modified such that one or more sensitizers is attached to the protein (e.g., attached to an amino acid). For example, any residue of the segment may be replaced with a sensitizer or any residue may be modified by attaching a sensitizer. For example, when the segment is the same length of LanM (full translated sequence of LanM), the 41^{st}, 62^{nd}, 65^{th}, 69^{th}, 87^{th}, 90^{th}, 94^{th}, 114^{th} residue, or a combination thereof of the segment is replaced with the sensitizer. In various other examples, the 4^{th}, 7^{th}, 11^{th} residues, or a combination thereof of one or more of the EF hands are replaced with a sensitizer residue. The aforementioned residue numbers refer to the residues in the full translated sequence of LanM, but still refer to the same residue in when the protein does not have the signal peptide. For example, the 41^{st} residue refers to the same threonine that is bolded in the following sequences:

(SEQ ID NO: 2)
APTTTTKVDIAAFDPDKDGTIDLKEALAAGSAAFDKLDPDKDGTLDAKE
LKGRVSEADLKKLDPDNDGTLDKKEYLAAVEAQFKAANPDNDGTIDARE
LASPAGSALVNLIR
and
(SEQ ID NO: 4)
MAFRLSSAVLLAALVAAPAYAAPTTTTKVDIAAFDPDKDGTIDLKEALA
AGSAAFDKLDPDKDGTLDAKELKGRVSEADLKKLDPDNDGTLDKKEYLA
AVEAQFKAANPDNDGTIDARELASPAGSALVNLIR.

Various sensitizers may be used. For example, the sensitizer is chosen from tryptophan, tryptophan analogs (e.g., 4-aza, 5-aza, and 7-aza-tryptophans; cyano-tryptophans; boron- and nitrogen-containing BN-tryptophan, and the like), naphthalimides, coumarins, acridones (e.g., acridon-2-ylalanine residues), other fluorophores, and the like, and combinations thereof. In various examples, the sensitizer residue is tryptophan. In various examples, the segment is LanM, where at least one (e.g., one) residue (e.g., the 41^{st}, 62^{nd}, 65^{th}, 69^{th}, 87^{th}, 90^{th}, 94^{th}, 114^{th} residue) is replaced with tryptophan. In various other examples, the protein is LanM, where at least one (e.g., one) residue (e.g., the 41^{st}, 65^{th}, 87^{th}, 90^{th}, 94^{th}, or 114^{th} residue) is replaced with tryptophan. In various examples, when the protein is LanM, the 90^{th} residue is tryptophan. In various other examples, a sensitizer is installed through cellular expression, in vitro protein/peptide synthesis, or via reaction with a cysteine or other nucleophilic residue on the protein or via an electrophilic position on the protein with a nucleophilic group on the sensitizer.

In various examples, a protein of the present disclosure has the following sequence:

(SEQ ID NO: 5)
MAFRLSSAVLLAALVAAPAYAAPTTTTKVDIAAFDPDKDGWIDLKEALA
AGSAAFDKLDPDKDGTLDAKELKGRVSEADLKKLDPDNDGTLDKKEYLA
AVEAQFKAANPDNDGTIDARELASPAGSALVNLIR;
(SEQ ID NO: 6)
MAFRLSSAVLLAALVAAPAYAAPTTTTKVDIAAFDPDKDGTIDLKEALA
AGSAAFDKLDPDKDGWLDAKELKGRVSEADLKKLDPDNDGTLDKKEYLA
AVEAQFKAANPDNDGTIDARELASPAGSALVNLIR;
(SEQ ID NO: 7)
MAFRLSSAVLLAALVAAPAYAAPTTTTKVDIAAFDPDKDGTIDLKEALA
AGSAAFDKLDPDKDGTLDAKELKGRVSEADLKKLDPDWDGTLDKKEYLA
AVEAQFKAANPDNDGTIDARELASPAGSALVNLIR;
(SEQ ID NO: 8)
MAFRLSSAVLLAALVAAPAYAAPTTTTKVDIAAFDPDKDGTIDLKEALA
AGSAAFDKLDPDKDGTLDAKELKGRVSEADLKKLDPDNDGWLDKKEYLA
AVEAQFKAANPDNDGTIDARELASPAGSALVNLIR;
(SEQ ID NO: 9)
MAFRLSSAVLLAALVAAPAYAAPTTTTKVDIAAFDPDKDGTIDLKEALA
AGSAAFDKLDPDKDGTLDAKELKGRVSEADLKKLDPDNDGTLDKWEYLA
AVEAQFKAANPDNDGTIDARELASPAGSALVNLIR;
(SEQ ID NO: 10)
MAFRLSSAVLLAALVAAPAYAAPTTTTKVDIAAFDPDKDGTIDLKEALA
AGSAAFDKLDPDKDGTLDAKELKGRVSEADLKKLDPDNDGTLDKKEYLA
AVEAQFKAANPDNDGWIDARELASPAGSALVNLIR;
(SEQ ID NO: 17)
MAFRLSSAVLLAALVAAPAYAAPTTTTKVDIAAFDPDKDGTIDLKEALA
AGSAAFDKLDPDWDGTLDAKELKGRVSEADLKKLDPDNDGTLDKKEYLA
AVEAQFKAANPDNDGTIDARELASPAGSALVNLIR;
(SEQ ID NO: 18)
MAFRLSSAVLLAALVAAPAYAAPTTTTKVDIAAFDPDKDGTIDLKEALA
AGSAAFDKLDPDKDGTLDAWELKGRVSEADLKKLDPDNDGTLDKKEYLA
AVEAQFKAANPDNDGTIDARELASPAGSALVNLIR;

or a sequence with at least 70% homology (e.g., at least 70%, at least 75%, at least 80%, at least 85%, at least 90%, or at least 95% homology). In various other embodiments, a protein of the present disclosure does not have the signal peptide portion of the protein. Example of such proteins include:

(SEQ ID NO: 11)
APTTTTKVDIAAFDPDKDGWIDLKEALAAGSAAFDKLDPDKDGTLDAKE
LKGRVSEADLKKLDPDNDGTLDKKEYLAAVEAQFKAANPDNDGTIDARE
LASPAGSALVNLIR;
(SEQ ID NO: 12)
APTTTTKVDIAAFDPDKDGTIDLKEALAAGSAAFDKLDPDKDGWLDAKE
LKGRVSEADLKKLDPDNDGTLDKKEYLAAVEAQFKAANPDNDGTIDARE
LASPAGSALVNLIR;
(SEQ ID NO: 13)
APTTTTKVDIAAFDPDKDGTIDLKEALAAGSAAFDKLDPDKDGTLDAKE
LKGRVSEADLKKLDPDWDGTLDKKEYLAAVEAQFKAANPDNDGTIDARE
LASPAGSALVNLIR;
(SEQ ID NO: 14)
APTTTTKVDIAAFDPDKDGTIDLKEALAAGSAAFDKLDPDKDGTLDAKE
LKGRVSEADLKKLDPDNDGWLDKKEYLAAVEAQFKAANPDNDGTIDARE
LASPAGSALVNLIR;
(SEQ ID NO: 15)
APTTTTKVDIAAFDPDKDGTIDLKEALAAGSAAFDKLDPDKDGTLDAKE
LKGRVSEADLKKLDPDNDGTLDKWEYLAAVEAQFKAANPDNDGTIDARE
LASPAGSALVNLIR;
(SEQ ID NO: 16)
APTTTTKVDIAAFDPDKDGTIDLKEALAAGSAAFDKLDPDKDGTLDAKE
LKGRVSEADLKKLDPDNDGTLDKKEYLAAVEAQFKAANPDNDGWIDARE
LASPAGSALVNLIR;
(SEQ ID NO: 19)
APTTTTKVDIAAFDPDKDGTIDLKEALAAGSAAFDKLDPDWDGTLDAKE
LKGRVSEADLKKLDPDNDGTLDKKEYLAAVEAQFKAANPDNDGTIDARE
LASPAGSALVNLIR;
(SEQ ID NO: 34)
APTTTTKVDIAAFDPDKDGTIDLKEALAAGSAAFDKLDPDKDGTLDAWE
LKGRVSEADLKKLDPDNDGTLDKKEYLAAVEAQFKAANPDNDGTIDARE
LASPAGSALVNLIR;

or a sequence with at least 70% homology (e.g., at least 70%, at least 75%, at least 80%, at least 85%, at least 90%, or at least 95% homology).

In various examples, a protein of the present disclosure has the following sequence:

(SEQ ID NO: 35)
MAFRLSSAVLLAALVAAPAYAAPTTTTKVDIAAFDPDKDGXIDLKEALA
AGSAAFDKLDPDKDGTLDAKELKGRVSEADLKKLDPDNDGTLDKKEYLA
AVEAQFKAANPDNDGTIDARELASPAGSALVNLIR;
(SEQ ID NO: 36)
MAFRLSSAVLLAALVAAPAYAAPTTTTKVDIAAFDPDKDGTIDLKEALA
AGSAAFDKLDPDKDGXLDAKELKGRVSEADLKKLDPDNDGTLDKKEYLA
AVEAQFKAANPDNDGTIDARELASPAGSALVNLIR;
(SEQ ID NO: 37)
MAFRLSSAVLLAALVAAPAYAAPTTTTKVDIAAFDPDKDGTIDLKEALA
AGSAAFDKLDPDKDGTLDAKELKGRVSEADLKKLDPDXDGTLDKKEYLA
AVEAQFKAANPDNDGTIDARELASPAGSALVNLIR;
(SEQ ID NO: 38)
MAFRLSSAVLLAALVAAPAYAAPTTTTKVDIAAFDPDKDGTIDLKEALA
AGSAAFDKLDPDKDGTLDAKELKGRVSEADLKKLDPDNDGXLDKKEYLA
AVEAQFKAANPDNDGTIDARELASPAGSALVNLIR;
(SEQ ID NO: 39)
MAFRLSSAVLLAALVAAPAYAAPTTTTKVDIAAFDPDKDGTIDLKEALA
AGSAAFDKLDPDKDGTLDAKELKGRVSEADLKKLDPDNDGTLDKXEYLA
AVEAQFKAANPDNDGTIDARELASPAGSALVNLIR;
(SEQ ID NO: 40)
MAFRLSSAVLLAALVAAPAYAAPTTTTKVDIAAFDPDKDGTIDLKEALA
AGSAAFDKLDPDKDGTLDAKELKGRVSEADLKKLDPDNDGTLDKKEYLA
AVEAQFKAANPDNDGXIDARELASPAGSALVNLIR;
(SEQ ID NO: 41)
MAFRLSSAVLLAALVAAPAYAAPTTTTKVDIAAFDPDKDGTIDLKEALA
AGSAAFDKLDPDXDGTLDAKELKGRVSEADLKKLDPDNDGTLDKKEYLA
AVEAQFKAANPDNDGTIDARELASPAGSALVNLIR;
(SEQ ID NO: 42)
MAFRLSSAVLLAALVAAPAYAAPTTTTKVDIAAFDPDKDGTIDLKEALA
AGSAAFDKLDPDKDGTLDAXELKGRVSEADLKKLDPDNDGTLDKKEYLA
AVEAQFKAANPDNDGTIDARELASPAGSALVNLIR;

or a sequence with at least 70% homology (e.g., at least 70%, at least 75%, at least 80%, at least 85%, at least 90%, or at least 95% homology), where X is a sensitizer as described herein (e.g., tryptophan analogs (e.g., 4-aza, 5-aza, and 7-aza-tryptophans; cyano-tryptophans; boron- and nitrogen-containing BN-tryptophan, and the like), naphthalimides, coumarins, acridones (e.g., acridon-2-ylalanine residues), other fluorophores, and the like). In various other embodiments, a protein of the present disclosure does not have the signal peptide portion of the protein. Example of such peptides include:

(SEQ ID NO: 43)
APTTTTKVDIAAFDPDKDGXIDLKEALAAGSAAFDKLDPDKDGTLDAKE
LKGRVSEADLKKLDPDNDGTLDKKEYLAAVEAQFKAANPDNDGTIDARE
LASPAGSALVNLIR;
(SEQ ID NO: 44)
APTTTTKVDIAAFDPDKDGTIDLKEALAAGSAAFDKLDPDKDGXLDAKE
LKGRVSEADLKKLDPDNDGTLDKKEYLAAVEAQFKAANPDNDGTIDARE
LASPAGSALVNLIR;
(SEQ ID NO: 45)
APTTTTKVDIAAFDPDKDGTIDLKEALAAGSAAFDKLDPDKDGTLDAKE
LKGRVSEADLKKLDPDXDGTLDKKEYLAAVEAQFKAANPDNDGTIDARE
LASPAGSALVNLIR;
(SEQ ID NO: 46)
APTTTTKVDIAAFDPDKDGTIDLKEALAAGSAAFDKLDPDKDGTLDAKE
LKGRVSEADLKKLDPDNDGXLDKKEYLAAVEAQFKAANPDNDGTIDARE
LASPAGSALVNLIR;
(SEQ ID NO: 47)
APTTTTKVDIAAFDPDKDGTIDLKEALAAGSAAFDKLDPDKDGTLDAKE
LKGRVSEADLKKLDPDNDGTLDKXEYLAAVEAQFKAANPDNDGTIDARE
LASPAGSALVNLIR;
(SEQ ID NO: 48)
APTTTTKVDIAAFDPDKDGTIDLKEALAAGSAAFDKLDPDKDGTLDAKE
LKGRVSEADLKKLDPDNDGTLDKKEYLAAVEAQFKAANPDNDGXIDARE
LASPAGSALVNLIR;
(SEQ ID NO: 49)
APTTTTKVDIAAFDPDKDGTIDLKEALAAGSAAFDKLDPDXDGTLDAKE
LKGRVSEADLKKLDPDNDGTLDKKEYLAAVEAQFKAANPDNDGTIDARE
LASPAGSALVNLIR;
(SEQ ID NO: 50)
APTTTTKVDIAAFDPDKDGTIDLKEALAAGSAAFDKLDPDKDGTLDAXE
LKGRVSEADLKKLDPDNDGTLDKKEYLAAVEAQFKAANPDNDGTIDARE
LASPAGSALVNLIR;

or a sequence with at least 70% homology (e.g., at least 70%, at least 75%, at least 80%, at least 85%, at least 90%, or at least 95% homology), where X is a sensitizer as described herein (e.g., tryptophan analogs (e.g., 4-aza, 5-aza, and 7-aza-tryptophans; cyano-tryptophans; boron- and nitrogen-containing BN-tryptophan, and the like), naphthalimides, coumarins, acridones (e.g., acridon-2-ylalanine residues), other fluorophores, and the like).

In various examples, the present disclosure also provides a protein containing the D35N substitution to inactivate metal ion binding to EF hand 1 without significantly altering the other metal binding sites.

In an aspect, the present disclosure provides devices. The device comprises one or more proteins of the present disclosures.

Various devices may comprise a protein of the present disclosure. Non-limiting examples of devices include filters, membranes, sensors, handheld detector, plate reader, fluorimeter, biosensors, in-line monitors, and the like.

In an aspect, the present disclosure provides kits. The kits may provide one or more proteins of the present disclosure and/or one or more devices of the present disclosure. The kit may include instructions for use of the proteins or devices.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

A method of using a protein and/or device of the present disclosure may be a method for binding one or more lanthanides and/or actinides in a sample. Binding may occur by contacting the sample with one or more proteins and/or devices of the present disclosure. The method may be performed on various types of samples. Examples of samples include, but are not limited to drinking water, wastewater, ground water, ash ponds, aqueous extract from contaminated soil, drainage (e.g., mine drainage, such as, for example, acidic mine drainage) or leachate (e.g., landfill leachate). In various other examples, the sample is a solid sample. The method may be applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

Various lanthanides (e.g., lanthanide ions) and/or actinides (e.g., actinide ions) may be bound by a protein and/or device. In various examples, any lanthanide except for La or Lu is detected. For example, the lanthanide is chosen from Tb, Eu, Dy, Sm, Nd, and ions thereof. In various examples, the lanthanide is Tb or an ion thereof. The bound lanthanides and/or actinides may be the same or different. The concentration of the lanthanide and/or actinides in the sample may be less than 100 ppm (e.g., less than 90, 80, 70, 60, 50, 40, 30, 20, 10, 1, 0.1, or 0.05 ppm).

In various examples, the one or more lanthanides and/or actinides bound to the one or more proteins and/or devices may be isolated from the proteins and/or devices and recovered. The lanthanides and/or actinides may be unbound by lowering the pH below ˜2.5 or by adding a chelator (e.g., citrate, EDTA, EGTA, or the like). The one or more proteins and/or devices may be reused after the one or more lanthanides are unbound and separated.

A method of the present disclosure may be a method of detecting and/or quantifying the amount of one or more lanthanides and/or actinides in a sample. The method may comprise contacting the sample with one or more proteins and/or device of the present disclosure. The contacted sample may then be exposed to light and the resulting emission of the exposed contacted sample. The resulting emission results may then be compared to a known standard curve for a specific lanthanide or actinide. The concentration may then be determined by that comparison. Known standard curves may be prepared based on the desire to detect and/or determine the quantity of any specific lanthanide or actinide. Methods of preparing standard curves are known in the art.

The method of detecting and/or quantified may be performed on various samples. Non-limiting examples of samples include drinking water, wastewater, ground water, ash ponds, aqueous extract from contaminated soil, drainage (e.g., mine drainage, such as, for example, acidic mine drainage) or leachate (e.g., landfill leachate). In various other examples, the sample is a solid sample. The method may be applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

Various lanthanides (e.g., lanthanide ions) and/or actinides (e.g., actinide ions) may be bound by a protein and/or device. For example, the lanthanide is chosen from Tb, Eu, Dy, Sm, Nd, and ions thereof. In various examples, the lanthanide is Tb or an ion thereof. The bound lanthanides and/or actinides may be the same or different. The concentration of the lanthanide and/or actinide in the sample may be less than 1 ppm.

In an aspect, the present disclosure provides a method to screen LanM variants using sensitized terbium luminescence, for altered metal ion selectivity (for separation applications). For example, a method uses a LanM containing a sensitizer (e.g., tryptophan at position 87, 90, or 94, or the equivalents in EF hand 2.

A LanM containing a sensitizer (e.g., tryptophan at position 87, 90, or 94, or the equivalents in EF hand 2 or EF1, EF4, or EF3), may be used as the basis for generating variants containing one or more amino acid substitutions in the protein, such as within, or near to, EF hands 2 and 3. A defined amount of terbium ions (e.g., 1 or 2 or 3 equivalents) may be added to each variant to give a luminescence signal. Then a competing ion (e.g., another lanthanide, or actinide, or other metal ion) can be added and the luminescence signal can be measured after a period of time, looking for the most (or least) efficient outcompetition of the sensitized terbium luminescence signal. Thereby, a comparison of the affinities of the protein for Tb versus any other metal ion can be made.

A method using the protein with the D35N substitution for any of the above applications.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements provide various embodiments of the present disclosure.

Statement 1. A protein having the sequence or comprising a segment having the sequence of Methylorubrum extorquens AM1 lanmodulin (LanM) or a truncated sequence thereof or sequence having 70%, 75%, 80%, 85%, 90%, or 95% homology to the sequence of LanM or of the truncated sequence thereof, wherein at least one amino acid residue of the protein or segment is replaced with a sensitizer or modified such that the sensitizer is attached to the protein or the segment.

Statement 2. A protein according to Statement 1, wherein when the segment has the sequence of LanM, the 41^{st}, 62^{nd}, 65^{th}, 69^{th}, 87^{th}, 90^{th}, 94^{th}, 114^{th} residue, or a combination thereof of the segment is replaced with the sensitizer, or the 4^{th}, 7^{th}, 11^{th} residue, or a combination thereof of an EF hand is replaced with the sensitizer.

Statement 3. A protein according to Statements 1 or 2, wherein the sensitizer is chosen from tryptophan, tryptophan analogs (e.g., 4-aza, 5-aza, and 7-aza-tryptophans; cyano-tryptophans; boron- and nitrogen-containing BN-tryptophan, and the like), naphthalimides, coumarins, acridones (e.g., acridon-2-ylalanine residues), other fluorophores, and the like, and combinations thereof.

Statement 4. A protein according to any one of the preceding Statements, wherein the sensitizer is tryptophan.

Statement 5. A protein according to any one of the preceding Statements, wherein the protein or the segment has the sequence of LanM with at least one residue replaced with the sensitizer.

Statement 6. A protein according to any one of the preceding Statements, wherein the 87^{th}, 90^{th}, or 94^{th} residue of the protein or the segment is replaced with tryptophan.

Statement 7. A protein according to any one of the preceding Statements, wherein the protein or the segment has the sequence of LanM and the 90^{th} residue of the protein or the segment is replaced with tryptophan.

Statement 8. A protein having the sequence or comprising a segment having the sequence of SEQ ID NO:2 or sequence having at least 80% homology thereto, wherein at least one amino acid residue of the protein or segment is replaced with a sensitizer or modified such that the sensitizer is attached to the protein or the segment.

Statement 9. A protein according to Statement 8, wherein the sensitizer is chosen from tryptophan, tryptophan analogs, naphthalimides, coumarins, acridones (e.g., acridon-2-ylalanine residues), and combinations thereof.

Statement 10. A protein according to Statement 8 or Statement 9, wherein the protein is any one of sequences SEQ ID NO:43-50 or a sequence having 80% homology thereto to any one of sequences SEQ ID NO:43-50.

Statement 11. A protein according to Statement 10, wherein the protein is any one of sequences SEQ ID NO:43-50.

Statement 12. A protein according to Statement 9, wherein the sensitizer is tryptophan.

Statement 13. A protein according to Statement 12, wherein the protein is any one of sequences SEQ ID NO:11-19 or 34 or a sequence having 80% homology thereto to any one of sequences SEQ ID NO: 11-19 or 34.

Statement 14. A protein according to Statement 13, wherein the protein is any one of sequences SEQ ID NO:11-19 or 34.

Statement 15. A protein according to Statement 14, wherein the protein has the sequence SEQ ID NO:19.

Statement 16. A device comprising a protein of any one of the preceding Statements.

Statement 17. A device according to Statement 16, wherein the device is a filter, membrane, sensor, handheld detector, plate reader, fluorimeter, biosensor, in-line monitor, or the like.

Statement 18. A kit comprising a protein of any one of Statements 1-15 or the device of Statements 16 or 17.

Statement 19. A method for binding one or more lanthanides and/or actinides comprising contacting a sample suspected of comprising the one or more lanthanides and/or actinides with one or more proteins according to any one of Statements 1-15 or a device according to Statements 16 or 17, wherein one or more lanthanides and/or actinides binds to the protein or device.

Statement 20. A method according to Statement 19, wherein the sample is drinking water, wastewater, ground water, ash ponds, aqueous extract from contaminated soil, drainage (e.g., mine drainage, such as, for example, acidic mine drainage) or leachate (e.g., landfill leachate), or solid sample.

Statement 21. A method according to Statements 19 or 20, wherein the one or more lanthanides are chosen from Tb, Eu, Dy, Sm, Nd, and ions thereof.

Statement 22. A method according to any one of Statements 19-21, wherein the lanthanide is Tb or an ion thereof.

Statement 23. A method according to Statements 19 or 20, wherein the one or more actinides are americium, curium, or an ion thereof.

Statement 24. A method according to any one of Statements 19-23, wherein the sample has a pH of 9 or below (e.g., 8.5 or below, 7.5 or below, 6.5 or below, 6 or below, 5.5 or below, 5 or below, 4.5 or below, 4 or below, 3.5 or below, or 3 or below).

Statement 25. A method according to any one of Statements 19-24, wherein the concentration of the one or more lanthanides and/or actinides is less than 100 ppm (e.g., less than 90, 80, 70, 60, 50, 40, 30, 20, 10, 1, 0.1. or 0.05 ppm).

Statement 26. A method according to any one of Statements 19-25, wherein the bound one or more lanthanides and/or actinides are unbound and isolated from the protein or device.

Statement 27. A method for detecting and quantifying one or more lanthanides and/or actinides in a sample, comprising contacting the sample with one or more proteins according to any one of Statements 1-15 or a device according to Statements 16 or 17; exposing the contacted sample with light; measuring a resulting emission of the exposed contacted sample; comparing the resulting emission to a known standard curve for a specific lanthanide; and determining the concentration of the specific lanthanide or actinide based on the comparison with the resulting emission and the known standard curve for the specific lanthanide or actinide, wherein the one or more lanthanides and/or actinides, if present, are detected and quantified based on the comparison of the resulting emission and the known standard curve.

Statement 28. A method according to Statement 27, wherein the sample is drinking water, wastewater, ground water, ash ponds, aqueous extract from contaminated soil, drainage (e.g., mine drainage, such as, for example, acidic mine drainage) or leachate (e.g., landfill leachate), or solid sample.

Statement 29. A method according to Statements 27 or 28, wherein the one or more lanthanides are chosen from Tb, Eu, Dy, Sm, Nd, and ions thereof.

Statement 30. A method according to any one of Statements 27-29, wherein the lanthanide is Tb or an ion thereof.

Statement 31. A method according to any one of Statements 27 or 28, wherein the one or more actinides is americium, curium, or a combination thereof.

Statement 32. A method according to any one of Statements 27-31, wherein the sample has a pH of 9 or below (e.g., 8.5 or below, 7.5 or below, 6.5 or below, 6 or below, 5.5 or below, 5 or below, 4.5 or below, 4 or below, 3.5 or below, or 3 or below).

Statement 33. A method according to any one of Statements 27-32, wherein the concentration of the one or more lanthanides and/or actinides is less than 100 ppm (e.g., less than 90, 80, 70, 60, 50, 40, 30, 20, 10, 1, 0.1, or 0.05 ppm).

Statement 34. A method to screen LanM variants comprising: contacting a defined amount of terbium ions with a LanM variant; measuring a luminescence signal; contacting the LanM contacted with terbium ions with a competing ion; and measuring the luminescence signal; and comparing the luminescence single.

Statement 35. A method according to Statement 34, wherein the competing ion is a lanthanide, actinide, or other metal ion.

The following examples are presented to illustrate the present disclosure. They are not intended to be limiting in any matter.

### Example 1

This example provides a description of a protein of the present disclosure and methods of making and using same.

Lanthanide f-f transitions are Laporte forbidden and therefore direct excitation is inefficient; this limitation may be overcome by incorporating a photosensitizer adjacent to the metal ion to absorb and transfer energy to the metal excited state (luminescence resonance energy transfer, LRET). The requirement for a nearby sensitizer is advantageous in that it enables probing of individual metal-binding sites utilizing chromophores (tyrosine or tryptophan) in the protein, either native or incorporated via site-directed mutagenesis.

Sensitized terbium luminescence was used to probe the mechanism of lanthanide recognition by this protein, as well as to develop a terbium-specific biosensor that can be applied directly in environmental samples. By incorporating tryptophan residues into specific EF hands, the order of metal binding of these three sites was inferred. Despite lanmodulin's remarkable lanthanide-binding properties, its coordination of two solvent molecules per site (by luminescence lifetime) and metal dissociation kinetics (k_{off}=0.02-0.05 s⁻¹, by stopped-flow fluorescence) are revealed to be rather ordinary among EF hands; what sets lanmodulin apart is that metal association is nearly diffusion limited (kₒₙ˜10⁹ M⁻¹ s⁻¹). Finally, it is shown that Trp-substituted lanmodulin can quantify as low as 3 ppb (18 nM) terbium directly in acid mine drainage at pH 3.2, in the presence of 100-fold excess of other rare earths and 100,000-fold excess of other metals, using a standard plate reader. These studies not only yield insight into lanmodulin's mechanism of lanthanide recognition and the structures of its metal-binding sites, but also show that the protein's unique combination of affinity and selectivity outperforms synthetic luminescence-based sensors, opening the door to rapid and inexpensive methods for selective sensing of individual lanthanides and actinides in the environment and in-line monitoring in industrial operations.

General considerations. Terbium(III) chloride hexahydrate (99.9%) and general laboratory chemicals for protein expression and purification and buffer preparation were obtained from Millipore Sigma. Deuterium oxide (99.9%) was purchased from Cambridge Isotope Laboratories. Primers were ordered from Integrated DNA Technologies (IDT). E. coli strains [5alpha, BL21(DE3)] for cloning and recombinant protein expression, respectively, as well as cloning reagents (Q5 DNA polymerase, OneTaq Quick-Load, KLD Enzyme Mix, DpnI) were purchased from New England Biolabs. Miniprep kits were from Omega Bio-tek. Protein gel electrophoresis was carried out using Invitrogen Novex WedgeWell 16% Tris-Glycine gels and a mini gel apparatus. Chelex 100 resin was purchased from BioRad. Automated protein chromatography was carried out on a GE Healthcare Biosciences Akta Pure fast protein liquid chromatography (FPLC) system. UV-visible absorption spectra were obtained on an Agilent Cary 60 UV-visible spectrophotometer using a quartz cuvette (Starna Cells). Well plates were analyzed using a BioTek Synergy H1 microplate reader. Fluorescence titrations and lifetime determinations were carried out on Cary Eclipse and PerkinElmer FL6500 spectrofluorometers, respectively, using a quartz septum cell micro fluorometer cuvette (10 mm pathlength, Starna Cells). Circular dichroism measurements were carried out in the X-ray Crystallography and Automated Biological Calorimetry Facility at Penn State using a 1-mm pathlength quartz CD cuvette (Jasco J/0556). Stopped flow UV-vis measurements were made on an Applied Photophysics SX20 spectrophotometer, equipped with a 450 long-pass filter (Corion LL-450-F-T539) and a fluorescence detector. All protein and metal solutions were made in 2 mL microcentrifuge tubes or 15 mL or 50 mL centrifuge tubes purchased from Sarstedt. All thermodynamic and kinetic data were analyzed, and curve fitting was performed, in Origin 2018.

Acid mine drainage (AMD). The AMD sample was collected from the feed of an AMD treatment facility operated by the Pennsylvania Department of Environmental Protection (Pennsylvania, USA). The source was from the lower Kittanning coal seam. The metal content of the sample was analyzed using inductively coupled plasma mass spectrometry (ICP-MS) on a Thermo Fisher Scientific ICAP RQ (ICP-MS) at the Penn State College of Earth and Mineral Sciences, Earth and Environmental Systems Institute, Laboratory for Isotopes and Metals in the Environment. The AMD sample was diluted 20χ into 2% HNO₃ (Aristar Ultra, BDH VWR Analytical), and a blank of 2% HNO₃ was subtracted from each analyte prior to elemental content determination. The pH of the sample was 3.24.

Construction, expression, and purification of Trp-substituted LanM (Trp-LanM) variants. Mutagenesis of T41, T65, N87, T90, K94, and T114 to W was performed on pET24a-LanM using the forward and reverse primers shown in Table 1, using NEB KLD enzyme mix. Reactions were run for 4 h at room temperature and then supplemented with 2 U/μL DpnI for 30 min at 37° C. prior to transformation. Transformants were screened for insert by colony PCR (OneTaq Quick-Load) and the correct insert was confirmed by DNA sequencing by Genewiz, yielding the plasmids in Table 2. The variants were expressed and purified as described previously for the wt protein (lysis, anion exchange, size exclusion chromatography), and stored in 20 mM MOPS, 100 mM KCl, 5 mM acetate, 5% glycerol, pH 7.0 (Buffer A).

TABLE 1
Primers used for cloning of Trp-LanM constructs.ª

| Name | Sequence |
|---|---|
| N87W-forward | 5′-GGACCCTGACTGGGACGGAACCC-3′ (SEQ ID NO: 20) |
| N87W-reverse | 5′-AGCTTCTTAAGGTCTGCC-3′ (SEQ ID NO: 21) |
| T90W-forward | 5′-CAATGACGGATGGCTGGACAAGAAAGAG-3′ (SEQ ID |
| | NO: 22) |
| T90W-reverse | 5′-TCAGGGTCCAGCTTCTTA-3′ (SEQ ID NO: 23) |
| K94W-forward | 5′-CCTGGACAAGTGGGAGTACTTAGCAGC-3′ (SEQ ID NO: 24) |
| K94W-reverse | 5′-GTTCCGTCATTGTCAGGG-3′ (SEQ ID NO: 25) |
| T41W-forward | 5′-CAAAGATGGGTGGATCGATCTGAAAGAGGCTTTGGCGG-3′ |
| | (SEQ ID NO: 26) |
| T41W-reverse | 5′-TCCGGGTCAAACGCCGCG-3′ (SEQ ID NO: 27) |
| T65W-forward | 5′-TAAAGATGGTTGGCTGGACGCCAAAGAG-3′ (SEQ ID |
| | NO: 28) |
| T65W-reverse | 5′-TCCGGGTCCAACTTGTCG-3′ (SEQ ID NO: 29) |
| T114W-forward | 5′-CAACGATGGCTGGATTGACGCCCG-3′ (SEQ ID NO: 30) |
| T114W-reverse | 5′-TCAGGGTTAGCGGCCTTA-3′ (SEQ ID NO: 31) |

Sequencing Primers

| | |
|---|---|
| T7P | 5′-TAATACGACTCACTATAGGG-3′ (SEQ ID NO: 32) |
| T7T | 5′-GCTAGTTATTGCTCAGCGG-3′ (SEQ ID NO: 33) |

^{a}Trp mutations are bolded

TABLE 2
Plasmids used in this study.

| Name | Notes |
|---|---|
| pET-24a | Km^{R} |
| pET-24a-LanM | Km^{R}, cytosolic expression |
| pET-24a-LanM-T41W | |
| pET-24a-LanM-T65W | |
| pET-24a-LanM-N87W | |
| pET-24a-LanM-T90W | |
| pET-24a-LanM-K94W | |
| pET-24a-LanM-T114W | |

Other methods of LanM purification also exist. For example, the protein could be secreted from the cell and collected from the culture, the thermal and acid stability of the protein could be exploited by treating the cells or lysate at high temperature (up to 95° C.) or low pH. Also, an ammonium sulfate fractionation may be used, e.g., precipitating most other cellular proteins with 40, 50, 60, etc. % (of saturated) ammonium sulfate, and then precipitating LanM with 100% (sat) ammonium sulfate. Other methods can also be envisioned.

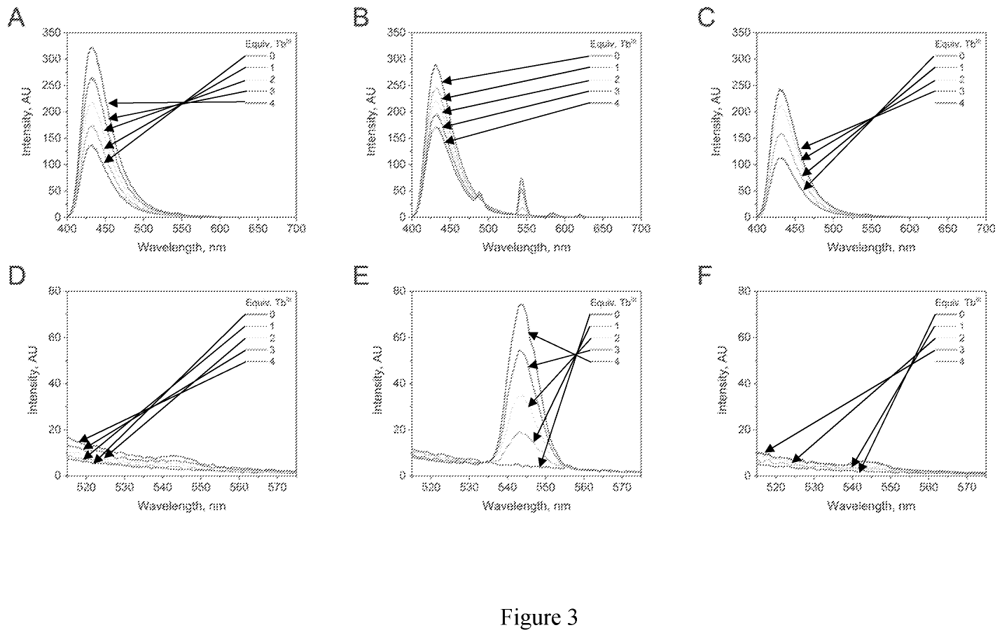

Determination of optimal Trp insertion points for LRET. Initial screening for the most suitable positions for Trp placement within LanM's EF hands utilized the EF3 Trp-substituted variants, N87W, T90W, and K94W. Stoichiometric LRET titrations of 20 μM protein in 20 mM MOPS, 100 mM KCl, 5 mM acetate, pH 7.0 (Buffer B) were carried out with a 2 mM solution of TbCl₃ in the same buffer. Titrations were performed on a Cary Eclipse fluorescence spectrophotometer using a 10 mm pathlength quartz septum cell micro fluorometer cuvette (Starna Cells) with the following instrument parameters: 280 nm excitation, 400-700 nm emission scan, 5 nm excitation and emission slit widths, 120 nm/min scan rate, 1 nm data interval, 250-395 nm excitation filter, 430-1100 nm emission filter, and high PMT voltage setting. A blank solution of buffer was subtracted from each spectrum prior to analysis and spectra were corrected for volume change prior to plotting. Substituting the seventh position in EF3 (T90W) revealed the greatest magnitude of LRET signal at 545 nm (FIG. 3), and this position was selected as the optimal position for Trp substitutions in each EF hand (T41W, T65W, T90W, T114W), from the perspective of signal intensity.

Circular dichroism spectroscopy on wt-LanM and Trp-LanM variants. Circular dichroism (CD) spectra of wt LanM and Trp-LanM variants were collected using a Jasco J-1500 CD spectrometer, thermostatted at 25° C., using a 1-mm pathlength quartz CD cuvette. Samples were scanned from 260-190 nm, with the following instrument settings: 1.00 nm bandwidth, 0.5 nm data pitch, 50 nm/min scan rate, 4 s average time. The cuvette contained 15 μM protein in 200 μL Chelex-treated 30 mM MOPS, 100 mM KCl, pH 7.2 (Buffer C), into which 1 to 5 equivalents TbCl₃ were titrated, and spectra were acquired. Three scans were acquired and averaged for each condition. A buffer blank spectrum was subtracted from each sample spectrum, and the spectra were corrected for volume change before plotting.

K_{d} determinations using CD spectroscopy. Solutions of Tb^{III} with free metal concentration buffered using ethylenediamine N,N′-disuccinic acid (EDDS) were prepared as described, in Buffer C. Protein was added to the low and high Tb^{III}-EDDS solutions separately to a final concentration of 15 μM, and EDDS solutions were mixed at various high:low ratios. The same ratio of high:low solutions were prepared without protein to yield the blank samples. Following a 1 hour (h) incubation at room temperature, samples were scanned from 260-210 nm, with the following instrument settings: 1.00 nm bandwidth, 0.5 nm data pitch, 50 nm/min scan rate, 4 s average time, 25° C. One accumulation was acquired for each condition. The blank spectrum acquired for each high:low ratio was subtracted from the corresponding Tb^{III}-LanM spectrum, and [θ]222 nm was plotted vs. free metal concentration.

Steady-state luminescence for K_{d},app determination on plate reader. EDDS-buffered solutions of Tb^{III} were prepared as described above. Protein was added to the low and high Tb^{III}-EDDS solutions separately to a final concentration of 10 μM, and EDDS solutions were mixed at various high:low ratios. Following a 1-h incubation at room temperature, time-resolved fluorescence emission was monitored from 400-700 nm on a BioTek Synergy H1 microplate reader in Greiner Cellstar 96-well half-area pClear plates with the following instrument settings: time-resolved delay 50 μs, collection time 1000 μs, fixed excitation at 295 nm, emission 400-700 nm with 1 nm steps, gain of 120, and read speed delay 200 ms. Data points were corrected for the significant contribution from emission of the Tb^{III}-EDDS complex, determined by running matching samples in the absence of protein. Data were analyzed by averaging the fluorescence emission at 544-546 nm and plotting against [Tb^{III}_{free}].

Luminescence lifetimes for q determination. Protein was diluted to 10 μM with 30 μM TbCl₃ in Buffer C. For D₂O containing samples, protein solutions (3 mL) were lyophilized overnight and resuspended in an equal volume of D₂O, the process was repeated, and then 10 μM protein in H₂O and 10 μM protein in D₂O were mixed in different proportions to achieve the % D₂O concentrations desired (0-75%). Lifetime measurements were obtained on a PerkinElmer FL 6500 Fluorometer with the following parameters: data mode phosphorescence (short), excitation correction off, source mode pulse, flash count 1, flash power 120 kW, frequency 50 Hz, excitation wavelength 295 nm, excitation slit 5 nm, excitation filter air, emission wavelength 545 nm, emission slit 5 nm, emission filter air, PMT voltage 700 V, PMT gain auto, emission correction off, response time 0.5 s, delay time 0 μs, gate time 20 ms. Three independent trials were performed for each condition, and the τ values determined from the fitted curves were averaged and plotted against % H₂O to determine τ(D₂O) from the y-intercept of the line. The values of q were calculated using the method of Horrocks (eq. 1), where A=5.0 ms.

$$q = A ( \frac{1}{τ ( {H}_{2} O )} - \frac{1}{τ ( {D}_{2} O )} ) \qquad ( eq . 1 )$$

Stopped-flow fluorometry. Stopped-flow fluorometry measurements were carried out at 25° C., maintained by a circulating water bath. One syringe contained a solution of 10 μM Trp-LanM and 30 μM TbCl₃, prepared in Chelex-treated Buffer C. The contents of this syringe were mixed in a 1:1 ratio with solutions of EGTA (10, 5, 2.5, and 1.25 mM, in Chelex-treated Buffer C) in a second syringe. Data were acquired with the following parameters: 1 mm slit width, 2 mm pathlength, with excitation at 295 nm, collecting 2000 data points over 120 s (T41W) or 200 s (T90W), and 12.5 μs sample period. Three shots were collected and averaged for each condition. Although a 450 nm long-pass filter was used, there was some residual Trp fluorescence in addition to the LRET signal in the emission channel. Curve fitting was performed in Origin 2018, fitted to either a single exponential (T41W) or a double exponential (T90W) decay.

Determination of limits of detection in plate reader luminescence assays. TbCl₃ stock solutions (10-50 μM) were made fresh each day in 20 mM acetate, 100 mM KCl, pH 5.0 (Buffer D). Protein samples were diluted to 1 μM or 10 μM in each of the following buffers: Buffer B (pH 7.0); Buffer D (pH 5.0); 20 mM acetate, 100 mM KCl, pH 4.0 (Buffer E); 20 mM ammonium formate, 100 mM KCl, pH 3.0 (Buffer F); and 20 mM ammonium formate, 100 mM KCl, pH 2.0 (Buffer G). Time-resolved luminescence emission was monitored from 400-700 nm (1 nm increments) in Greiner BioOne 96-well white flat-bottom Lumitrac plates with the following instrument settings: time-resolved delay 200 μs, collection time 1000 μs, fixed excitation at 280 nm, gain of 140, and read speed delay 100 ms. A blank containing buffer and Tb was subtracted from each corresponding spectrum prior to data analysis. Data were analyzed by averaging the emission at 544-546 nm from three independent replicates and plotting against Tb concentration (0.8 ppb to 35.8 ppb, or 5 to 225 nM). Limits of detection were calculated from the slope (m) of the regression line (Origin 2018) and the standard deviation (s) of the sample with the lowest Tb concentration that displayed a peak across the entire pH range (2.4 or 15.9 ppb, for T90W and T41W, respectively), according to eq. 2.

$$LOD = \frac{3 s}{m} \qquad ( eq . 2 )$$

Quantification of Tb in AMD using T90W-LanM. Time-resolved luminescence emission was monitored from 400-650 nm in Greiner BioOne 96-well white flat-bottom Lumitrac plates with the same instrument settings as above. The sample volume was 200 μL. With these settings, there was no significant emission from the AMD in the blank sample (without protein). For terbium detection, T90W-LanM was added to a concentration of 10 μM, from a 1 mM stock in Buffer A. Addition of the protein did not significantly affect pH of the AMD. Tb concentration was determined by averaging the emission at 544-546 nm. To enable Tb quantification, a standard curve was generated by mixing the same volumes of AMD and protein as above but with 0.4-2 μL of 2.5 ppm Tb^{III} in Buffer D added, to yield 5-25 ppb Tb. The data with 0, 5, 10, 15, 20, and 25 ppb Tb added were fitted to a regression line, and the emission of the sample without Tb added was divided by the slope of the line, yielding the estimated Tb concentration.

### Results and Discussion

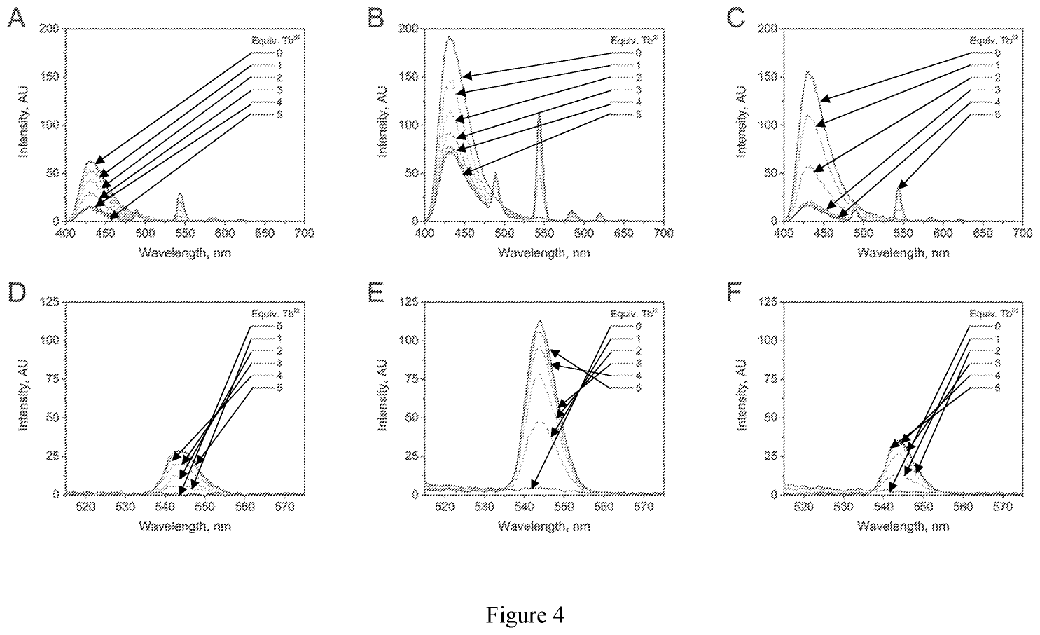

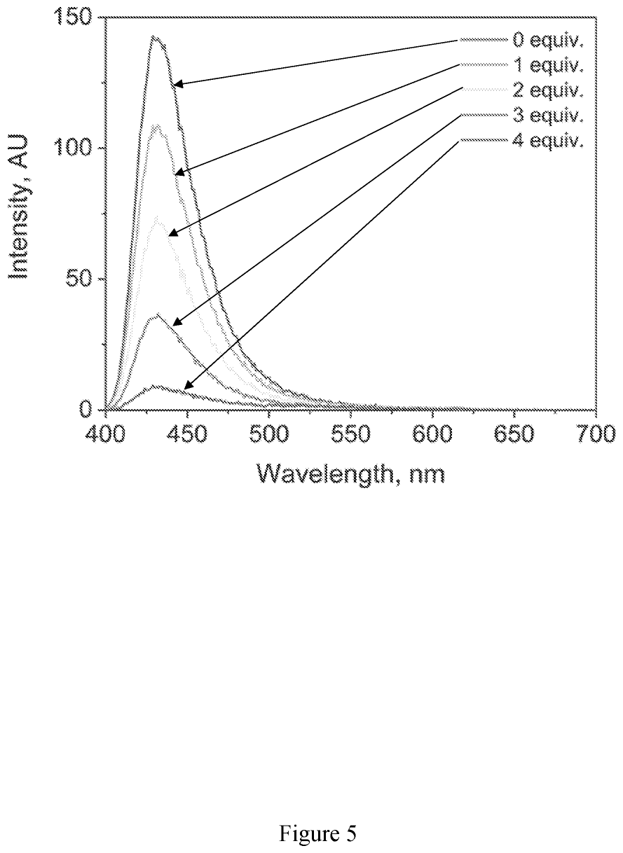

Determination of optimal Trp insertion points in LanM. LanM possesses no Trp residues natively, facilitating the strategy to site-specifically probe metal binding using sensitized luminescence. On the basis of the NMR solution structure of Y^{III}-bound LanM, three positions were selected in EF3-N87 (4^{th} position), T90 (7^{th} position), and K94 (11^{th} position)—for Trp substitution and preliminary assays of energy transfer efficiency. It was hypothesized that these substitutions would minimally interfere with metal ion binding yet also be sufficiently close to the Tb^{III} ion to yield a robust LRET signal. These variants were screened by stoichiometric Tb^{III} titrations (pH 7.2), exciting the Trp at 280 nm and monitoring in-growth of the luminescence spectrum of Tb^{III}, in particular the most intense feature at ˜545 nm (FIG. 3). In the case of N87W and K94W, Trp emission increased with addition of Tb^{III}, indicative of a change in environment of the fluorophore, presumably associated with the metal-induced conformational change. However, LRET was weak in these variants. Meanwhile, T90W showed the expected quenching of the Trp emission accompanied by strong Tb^{III} emission. Slight differences in Tb^{III}-binding stoichiometry were observed with these variants. Three equiv. Tb^{III} was sufficient to maximize Trp response and LRET signal for K94W but not other variants (4 equiv.); by comparison, wild-type LanM binds 3 equiv. REEs with high affinity. Therefore, K94W may introduce the least perturbation into EF3, but it gives a poor LRET signal. As a result, these preliminary studies suggested that the 7^{th} position Thr residue was the most promising position for Trp substitution from the perspective of LRET efficiency. This result is consistent with characterization of other Trp-substituted EF-hand proteins including calmodulin and synthetic Tb^{III}-binding peptides, including LBTs. Therefore, the 7^{th} positions of the other three EF hands were substituted with Trp (T41W, T65W, T114W). All of these constructs exhibited robust Tb^{III} emission (FIG. 4), whereas energy transfer to Eu^{III} occurred, but with minimal emission, as expected based on other protein systems (FIG. 5). Presumably, the response of T114W (EF4) reflects energy transfer to the Tb^{III} bound in EF1.

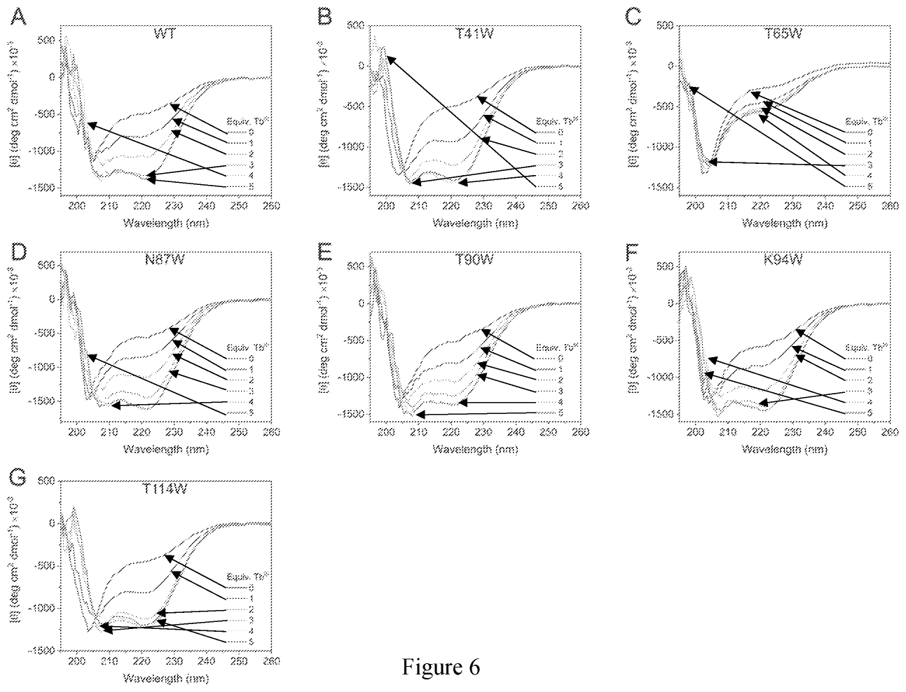

The Trp-substituted LanM variants (Trp-LanMs) were evaluated by CD spectroscopy to determine whether the Trp residue affected the apparent dissociation constant (K_{d,app}) and magnitude of the Tb^{III}-induced conformational change. All variants exhibited the same overall conformational change as the wild-type LanM (˜2.5-3-fold increase in the molar ellipticity at 222 nm, indicating increased helicity in the presence of Tb^{III} ions), with the notable exception of the EF2 insertion, T65W, which displays less helicity in the apoprotein as well as a nearly completely disrupted conformational response (FIG. 6). As a result of the substantial perturbation introduced by the Trp residue in EF2, this site was not directly probed further, although T90W in EF3 does appear to report on metal binding to EF2 as well (vide infra). Disruptive effects resulting from substitution of Trp residues at the EF-hand 7^{th} position has been observed previously for some Ca^{II}-binding EF-hand proteins. The other variants displayed full conformational response to 3 equiv. Tb^{III}, with the exception of N87W and T90W, which required four equivalents, similar to the fluorescence result (FIG. 3).

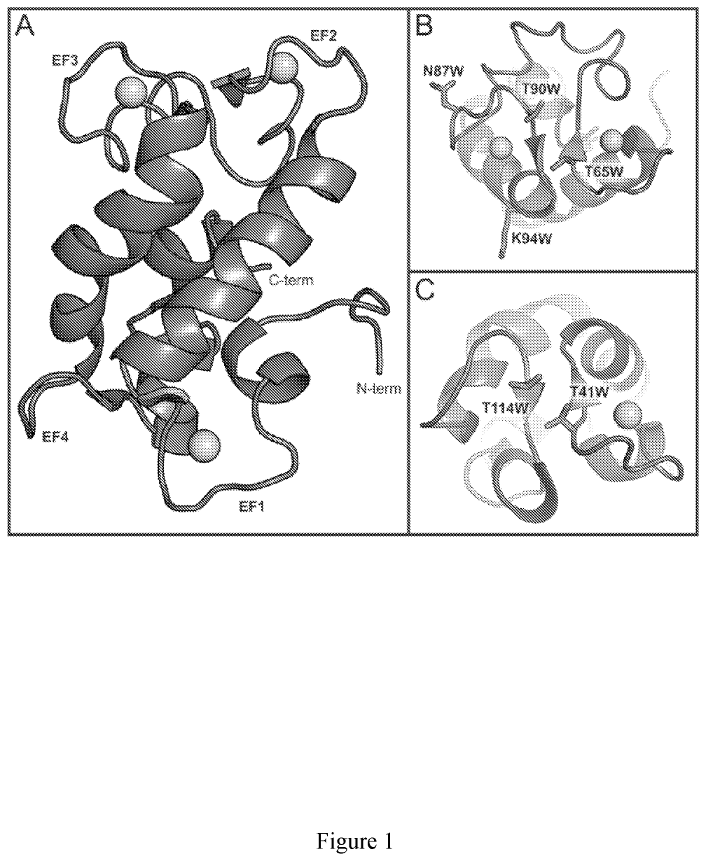

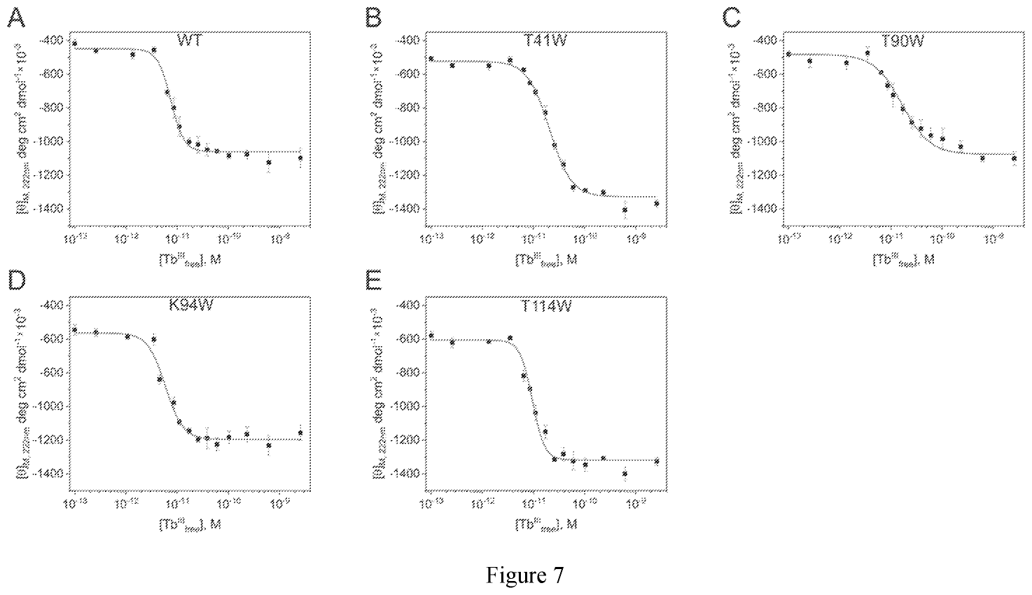

K_{d,app} values were determined for the Tb^{III}-bound Trp variants in comparison to wt LanM (FIG. 7, Table 3). Note that the K_{d,app} value for untagged wt LanM is slightly lower (7 μM) than for the C-terminally His-tagged protein (21 μM). The substitutions most distal to metal-binding sites (K94W in EF3 and T114W in EF4, which does not bind a metal under these conditions) displayed K_{d,app} values and Hill coefficients (n) very similar to wt LanM. T41W (EF1) displayed a slight increase in K_{d,app} and decrease in n. By contrast, T90W was disruptive, appearing to break the conformational response into two phases with K_{d,app}values ˜10 μM and ˜100 μM; however, two-phase fits did not converge well, so the single-phase fit is presented herein. Therefore, T41W and Ti 14W are both suitable probes of metal binding to EF1, and K94W is the least disruptive probe for EF3, although its very weak LRET intensity could limit some applications. The observation that T65W and (to a lesser extent) T90W, which are both at the interface of EF2 and EF3 (FIG. 1), are the most disruptive substitutions also provides important insights into LanM function. First, the failure of T65W-LanM to adopt the full helical structure of the wt protein suggests that EF2 is particularly critical for LanM's conformational change. Second, communication between EF2 and EF3 appears to be important for maintaining high Tb^{III} affinity overall, as suggested by characterization of T90W.

TABLE 3
Apparent K_{d} values and Hill coefficients (n) for Trp-LanMs,
determined using CD spectroscopy and luminescence methods,
and monitored at various EDDS-buffered free Tb^{III} ion concentrations.
Uncertainties represent mean ± SD for 3 independent experiments.

| | Circular dichroism titrations | | Luminescence titrations | |
|---|---|---|---|---|
| Construct | K_{d, app} (pM) | n | K_{d, app} (pM) | n |
| WT | 7.3 ± 0.9 | 2.2 ± 0.5 | NA^{a} | NA |
| T41W (EF1) | 22 ± 1 | 1.9 ± 0.2 | 42 ± 3 | 1.3 ± 0.1 |
| T90W (EF3) | 17 ± 3 | 1.3 ± 0.2 | 66 ± 8 | 0.8 ± 0.1 |
| K94W (EF3) | 6.1 ± 0.6 | 2.7 ± 0.5 | 6.2 ± 2.9 | 2.2 ± 0.6 |
| T114W (EF4) | 10 ± 1 | 2.1 ± 0.4 | 15 ± 1 | 1.5 ± 0.2 |

^{a}NA: not applicable

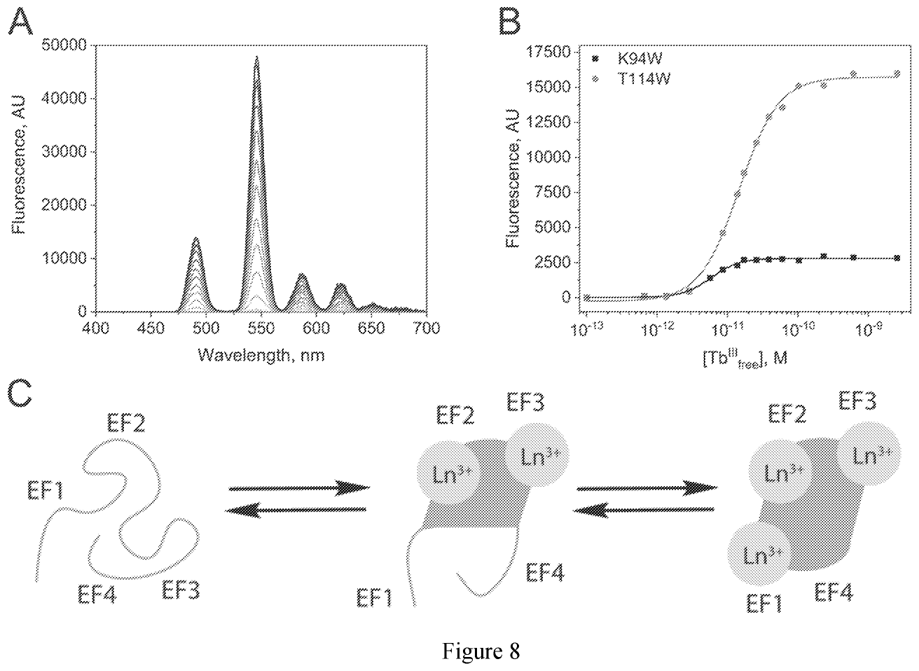

Steady state luminescence elucidates cooperative linkages between LanM's EF hands. Having characterized the overall conformational responses of the Trp-LanM variants, we next exploited the long-lived luminescence of Tb^{III} (FIG. 8A) using time-resolved detection to probe the individual binding site(s) in the vicinity of each Trp residue. It was reasoned that comparison of each of the extracted K_{d,app} values and cooperativities (Hill coefficient, n) to the CD-derived values for the whole protein could enable deduction of the order of metal binding and connectivity between the three EF hand binding sites.

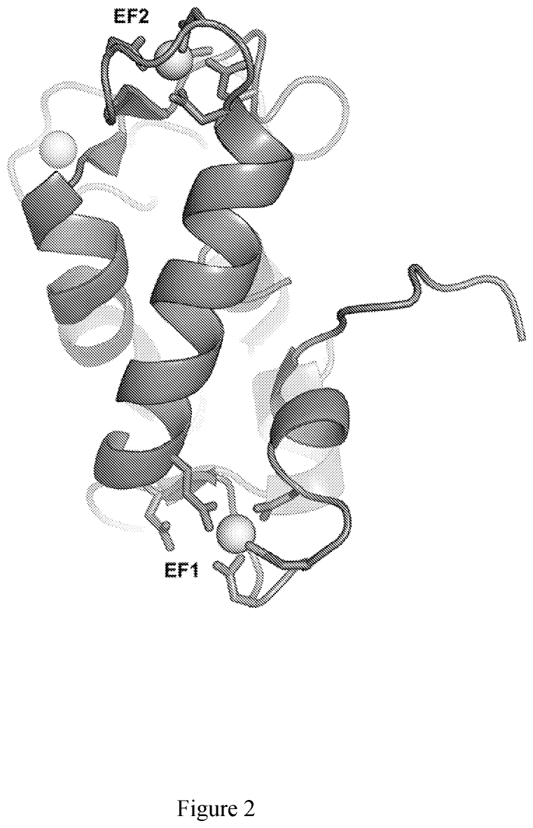

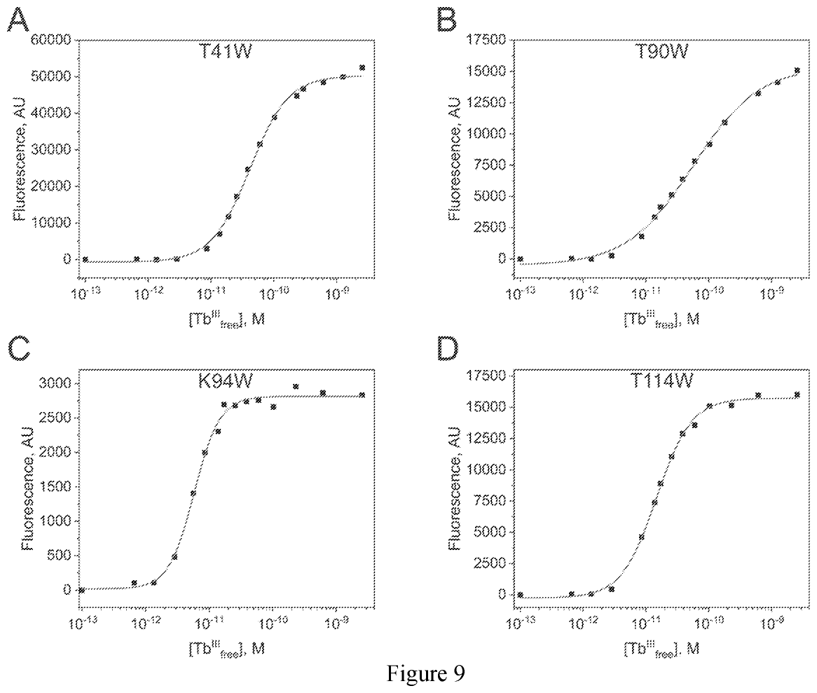

Overall, T41W and T90W showed the largest LRET response (FIG. 9), although all four constructs studied had sufficient responses to allow determination of apparent K_{d} values and Hill coefficients. T41W and T114W, which both report on Tb^{III} binding to EF1 (FIG. 1), exhibited lower Hill coefficients and slightly higher K_{d,app} values as determined by the LRET titrations, compared to the CD titrations (Table 3). Because the CD titration of T114W is least perturbed from wt values, this variant likely serves as the better reporter of EF1 binding; therefore, the K_{d,app} of EF1 is likely ˜15 μM (the value derived from LRET), slightly weaker than the main response. These observations suggest that metal binding to EF1 does not account for the main conformational response of LanM (i.e., it is the weakly cooperative second phase). However, because the Hill coefficients associated with the probes of EF1 binding are not exactly equal to 1, it is possible that metal binding to EF1 is slightly influenced to the main binding event (vide infra), perhaps via the helical connectivity between EF1 and EF2 (FIG. 2).

Consistent with the CD titrations, T90W is clearly a disruptive position for Trp placement within EF3, as revealed by a Hill coefficient, n≈1, indicating a lack of cooperativity, and a high K_{d} as compared to wt. Interestingly, the K_{d,app} value derived from the LRET data approximately corresponds to the apparent second phase in the CD titrations, with K_{d,app}-100 μM. These perturbations meant it was difficult to draw conclusions about cooperativity of metal binding using this variant. Therefore, the K94W variant, which displayed fully wt behavior in CD titrations, was instead used to investigate Tb^{III} binding to EF3. Although the LRET intensities observed with this variant were low (FIG. 8B, FIG. 3C), the apparent K_{d} was identical to the values determined by CD for this variant as well as for the wt, suggesting that EF3 is involved in LanM's primary metal-induced conformational change. Despite the fact that this Trp residue is predicted to be being much closer to the Tb^{III} bound in EF3 (˜9 Å) than to any other EF hand (e.g., ˜18 Å from EF2), and therefore likely only reports on EF3, the Hill coefficient was ˜2, indicating positive cooperativity between Tb^{III} binding to EF3 and to at least one other EF hand. Because EF1 is ruled out from the above considerations, EF3 must be communicating with EF2, supporting the conclusion from the above.

Together, these data support the model for metal binding and conformational change presented in FIG. 8C. Metal binding events in EF2 and EF3 display strong positive cooperativity and together are responsible for the major conformational change in the protein, accounting for ˜⅔ of its helical content. Metal binding to EF1 is slightly weaker and only weakly connected to binding at the other sites, and it is responsible for the remaining ⅓ of the protein's helical content.

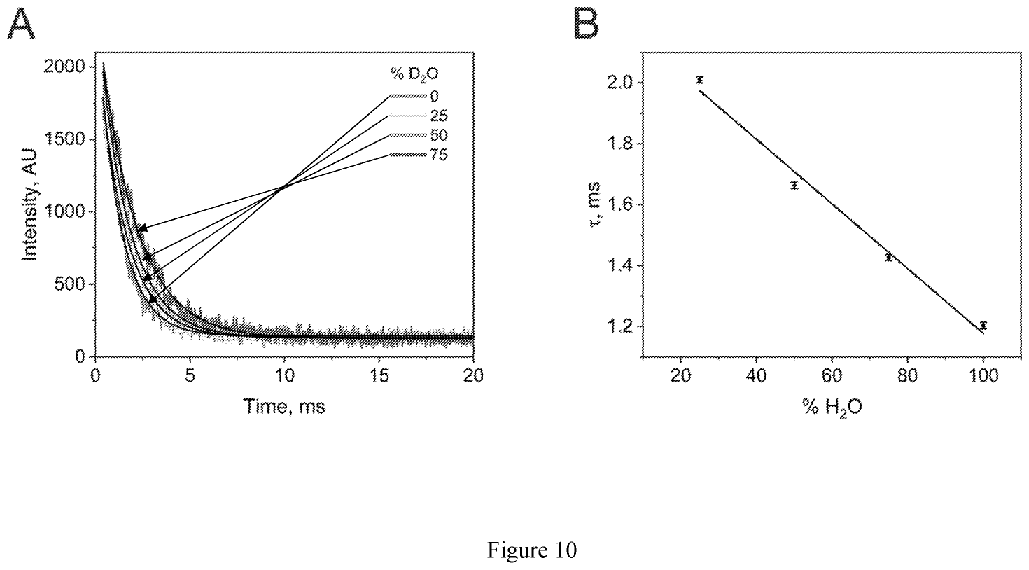

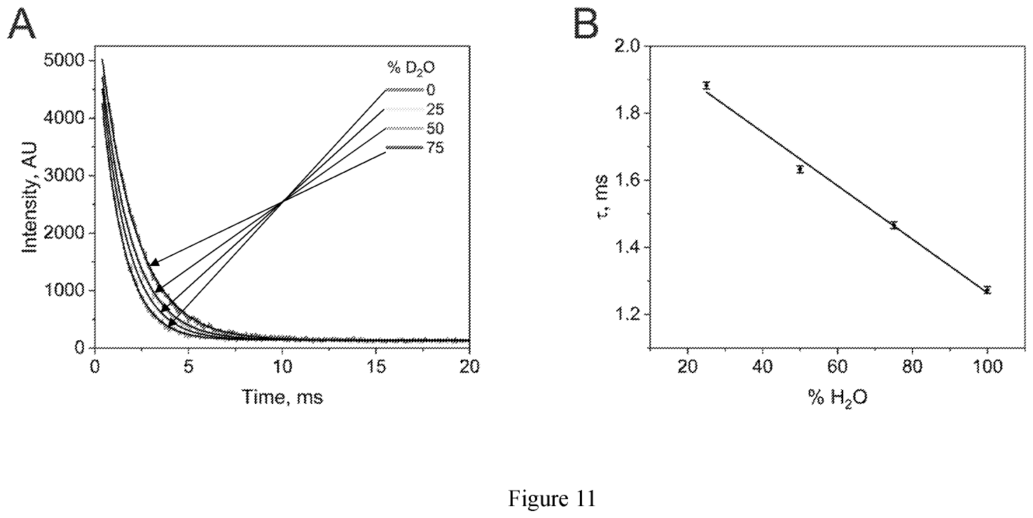

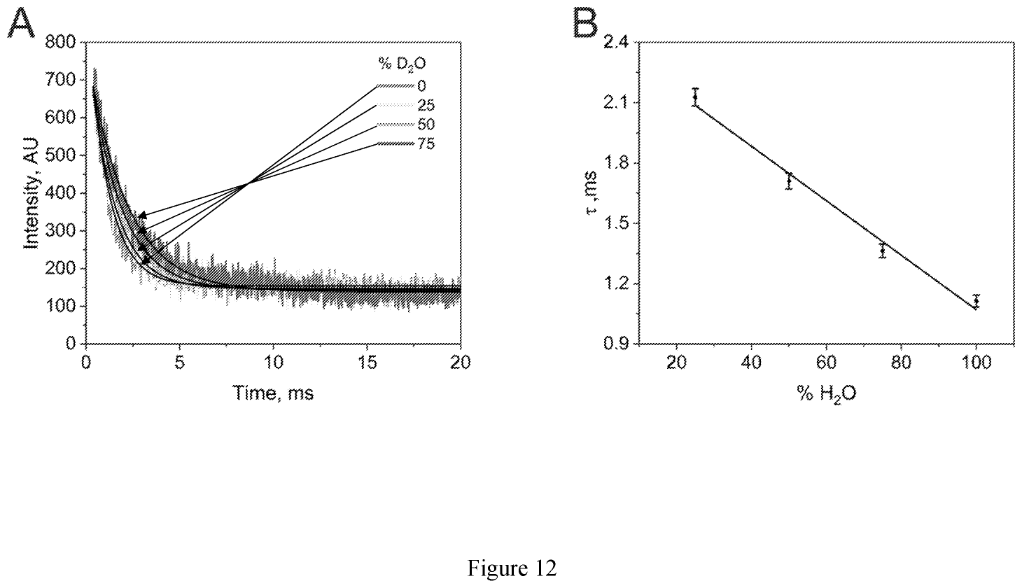

Luminescence lifetimes to investigate coordinated waters. Another critical aspect of understanding LanM's function is the structure of the metal binding sites. The determination of the NMR structure of Y^{III}-bound LanM was unable to determine whether protein residues saturated the metal-binding sites, or whether solvent molecules filled part of the coordination spheres. This information can be obtained from the lifetime of the Tb^{III} excited state, which is sensitive to the presence of coordinated water molecules due to the radiationless decay of the excited state via O—H vibrations. Because this decay pathway is suppressed in D₂O, the empirical relationship between the difference in decay rate constants (τ⁻¹) in the presence of H₂O and D₂O has been shown to yield the approximate number of coordinated water molecules (q). Therefore, LanM was probed using this method, utilizing the T41W and T90W variants because of their strong luminescence and representation of both pairs of EF hands. In both cases, the decays of the luminescence signals could be fitted to single exponentials (FIG. 10 for T41W and FIG. 11 for T90W). By varying the mole fraction of D20 in the protein solution, the q value determined for T41W is 2.1±0.1, and 1.7±0.1 for T90W. These results suggest that two solvent molecules are coordinated at both EF1 and EF3 metal-binding sites. To confirm the result for EF3, because of the perturbations introduced by the T90W substitution, measurements on K94W were also carried out, despite its much lower luminescence; these experiments also yielded a q value of ˜2 (2.4±0.1, FIG. 12). The presence of water molecules coordinated to Tb^{III} or Eu^{III} ions used to probe metal binding in Ca^{II}-binding EF hands proteins is common; for example, luminescence studies have found q=2 for Ln_{III}-bound metal sites in calmodulin and q=1 in parvalbumin. Therefore, despite the protein's unique affinity and selectivity for lanthanides, lanmodulin's metal sites are rather typical among EF-hand proteins with regard to solvent coordination. This result raises the question of whether the additional, conserved carboxylate residues at position 9 of LanM's EF hands are in fact coordinated, as originally proposed, or perhaps involved in hydrogen bonding with a coordinated water molecule, as residues at this position sometimes are in Ca^{II}-binding EF hands. Additionally, because the presence of coordinated solvent would be expected to increase the rate constant for metal ion dissociation, we next sought to use stopped flow spectrofluorometry to probe the kinetics of the system in order to better understand this selectivity.

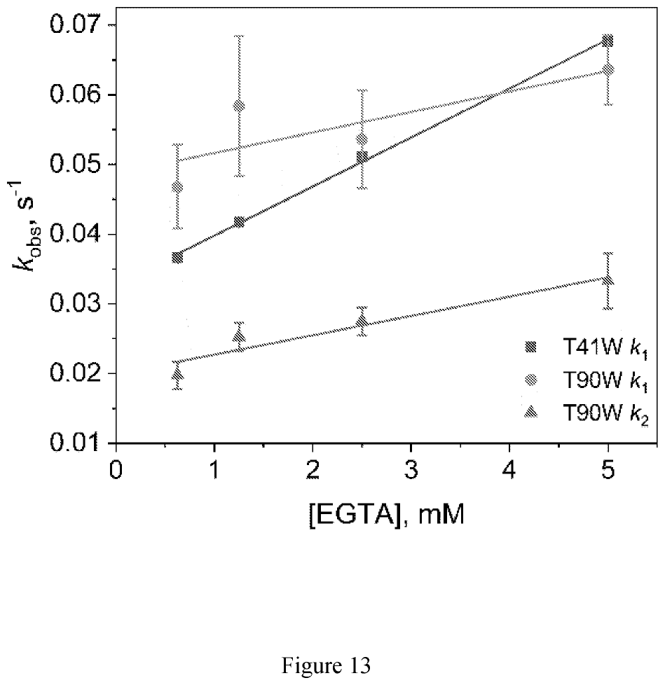

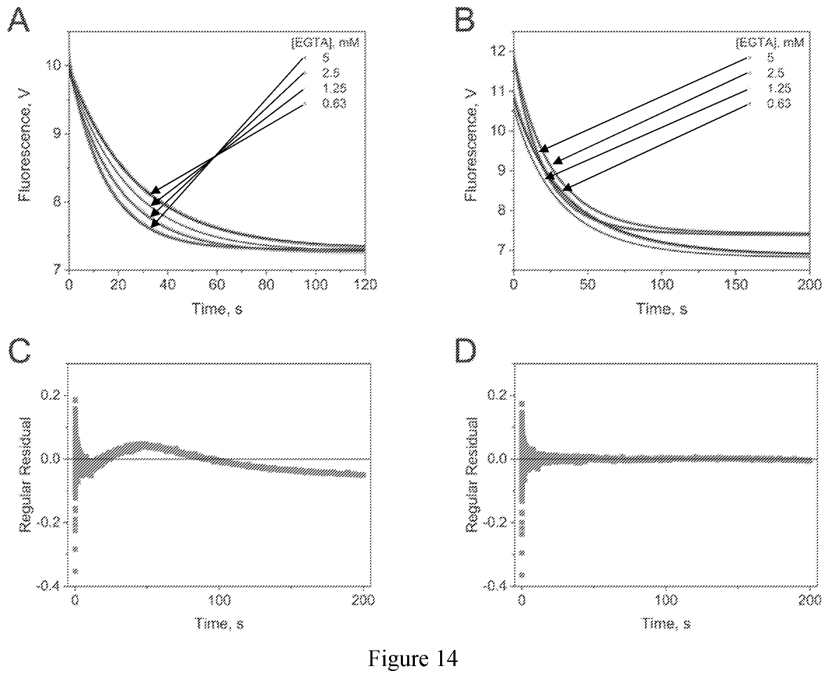

Probing kinetics of Tb(III) binding using stopped-flow spectrofluorometry. LanM's remarkable affinity for lanthanides may be explained by either a faster rate of metal association, slower rate of metal dissociation, or both, relative to other EF-hand proteins. The large luminescence changes associated with Tb^{III} binding to the T41W and T90W LanM variants, despite the perturbations in apparent K_{d}, allowed us to use stopped-flow spectrofluorometry to investigate the kinetics of site-specific Tb^{III} dissociation from LanM. In these experiments, the Trp-LanM variants were pre-loaded with 3 equivalents Tb^{III} and rapidly mixed with solutions of ethylene glycol-bis(p-aminoethyl ether)-N,N,N′,N-tetraacetic acid (EGTA) at four different concentrations and decay of the fluorescence signal above 450 nm (primarily the Tb^{III} luminescence) was monitored. Plotting of the decay rate constants (k_{obs}) at each EGTA concentration and extrapolation of the line to zero EGTA enables estimation of the dissociation rate constant (k_{off}) in the absence of chelator (FIG. 13). The T41W fluorescence decays could be fitted to single exponentials, resulting in a k_{off} of 0.033±0.001 s⁻¹. By contrast, fitting the T90W decays to a single exponential did not yield acceptable residuals (FIG. 14); fitting to two exponential phases was necessary, resulting in k_{off} values of 0.049±0.005 s⁻¹ and 0.020±0.002 s⁻¹ (Table 4). The requirement for two phases to fit the T90W data may reflect the position of this Trp residue between EF3 and EF2, allowing communication to each EF hand; the failure to distinguish a second phase in the luminescence decay experiments above may reflect either the lower signal to noise in the decay experiment or identical solvent coordination for both metal-binding sites. In support of the ability of Trp residues at the 7^{th} position to communicate with both EF hands in the pair, the T114W (EF4) variant exhibits LRET when Tb^{III} is bound in EF1 (FIG. 9, Table 3). Therefore, it is suggested that the two phases of the stopped flow data report on metal dissociation from EF2 and EF3, respectively, but the assignment of each phase to a particular EF hand was unable to be performed. It is speculated that the faster k_{off} may be associated with EF3, based on the influence of Trp90 on apparent K_{d}s measured by CD and steady-state LRET (Table 3). Of course, such perturbations from introduction of Trp residues may mean that the true k_{off} values for the wt protein may be somewhat smaller than those determined here. Nevertheless, these data suggest that the three metal-binding EF hands in LanM have relatively similar dissociation rate constants, on the order of 0.02-0.05 s⁻¹.

Even though LanM exhibits several orders of magnitude higher affinities for Ln^{III} ions than most other EF-hand proteins, LanM's k_{off} values are within the typical range for Tb^{III} dissociation from the proteins in this family (e.g., 0.05 and 0.5 s⁻¹ for parvalbumin and 0.01 s⁻¹ for galactose binding protein). The similarity of these values may be accounted for by the similar numbers of coordinated solvent molecules, as determined above. From the k_{off} values and K_{d,app} values (Table 3), both determined based on LRET, kₒₙ was estimated to be ˜8×10⁸ M⁻¹ s⁻¹ for T41W and 3-7×10⁸ M⁻¹ s⁻¹ for T90W (in the case of T90W, a single K_{d,app} value from the LRET titrations but two different k_{off} values prevents calculation of a single kₒₙ). These rate constants are very close to the diffusion limit, which is on the order of 10⁹-10¹⁰ M⁻¹ s⁻¹. Such rapid association kinetics may help to account for LanM's ability to selectively bind lanthanides even in complex solutions, with hundreds of millions-fold excess of competing metal ions. The optimization of LanM's kₒₙ values for lanthanide binding appears to be a key feature of the protein's metal recognition, whereas its dissociation rates are rather typical among EF-hand proteins. However, control of k_{off} may still be important; the observation that kₒₙ is near-optimal even for Tb^{III}, whereas LanM has even higher affinity for the early Ln^{III} ions, suggests that k_{off} differences may govern LanM's selectivity within the lanthanide series.

TABLE 4
Kinetic parameters from the fitted stopped-flow spectrofluorometry data. Experimental curves were
fitted to a single exponential decay (T41W) and a double exponential decay (T90W, see FIG. 14C).

| [EGTA], | T41W | | T90W | | | |
|---|---|---|---|---|---|---|
| mM | k₁ | Amplitude | k₁ | Amplitude | k₂ | Amplitude |
| 5.00 | 0.0676 ± 0.0002 | 2.744 ± 0.003 | 0.064 ± 0.005 | 2.6 ± 0.2 | 0.033 ± 0.004 | 1.9 ± 0.2 |
| 2.50 | 0.0511 ± 0.0002 | 2.642 ± 0.003 | 0.054 ± 0.007 | 2.2 ± 0.3 | 0.027 ± 0.002 | 2.3 ± 0.2 |
| 1.25 | 0.0417 ± 0.0001 | 2.628 ± 0.003 | 0.06 ± 0.01 | 1.1 ± 0.2 | 0.025 ± 0.002 | 2.7 ± 0.2 |
| 0.63 | 0.0366 ± 0.0001 | 2.617 ± 0.004 | 0.047 ± 0.006 | 1.6 ± 0.2 | 0.020 ± 0.002 | 2.3 ± 0.2 |

Trp-LanMs exhibit low limits of detection over a wide pH range. Whereas use of Trp-LanMs to characterize mechanism, structure, and kinetics of LanM was carried out at pH 7.2, potential broader application of these proteins as sensors would require responsiveness under a range of conditions, particularly in the presence of other metal contaminants and at low pH. Although LanM can selectively and quantitatively extract REEs from low-grade feedstocks containing only 30 ppm (˜200 μM) total REEs, environmental samples such as AMD typically harbor much lower concentrations, <1 ppm REEs, and Tb at only low ppb levels. Meanwhile, both these applications and monitoring of industrial processes necessitate robust performance at lower pHs than previous LaMP1 sensor could provide. With an eye toward these applications, the pH dependence and limits of detection (LODs) of Trp-LanM luminescence were determined.

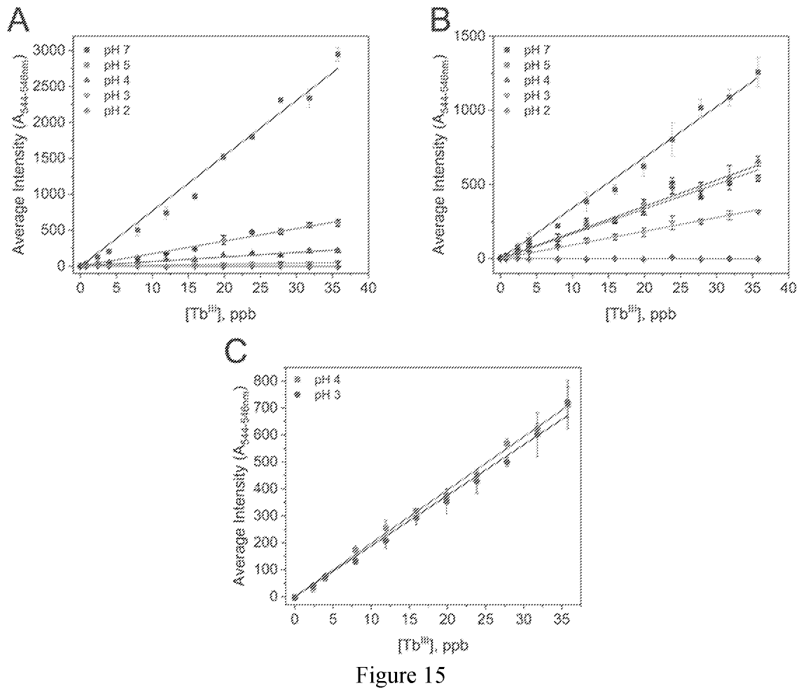

The T41W and T90W variants were used for these experiments, as they exhibited the greatest sensitized luminescence intensity of the Trp-LanM constructs characterized. Standard curves were generated with Tb^{III} concentrations ranging 0.8-36 ppb and between pH 2 and pH 7, in the presence of 1 or 10 μM of each protein. Given the long luminescence lifetime observed (FIG. 13), samples were detected in time-resolved luminescence mode, on a standard plate reader. At pH 3-7, linear responses were observed for both proteins (FIG. 15). Neither protein responded at pH 2. Because REEs have been shown to desorb from the protein at pH ˜2.5, this result reinforces that the observed luminescence signal is specific to interaction with LanM. Whereas at 1 μM, T90W-LanM exhibits a lower slope at pH 3 than at pH 4-5 (FIG. 15B), increasing the concentration to 10 μM results in constant slopes at pH 3 and 4 (FIG. 15C), suggesting that the metal is essentially fully bound to protein under these conditions. Interestingly, although T41W performed better than T90W at pH 7, its luminescence declined more quickly at lower pH values than that of T90W. Because wt LanM retains binding of 3 equivalents of REEs even down to pH 3, this decline is unlikely to result from metal dissociation. Instead, it is noted that the magnitude of the conformational response measured by CD decreases slightly at lower pH values. Together, even though the K_{d,app} of EF1 is similar to that of EF2/3 at pH 7.2 as demonstrated by our LRET studies (Table 3), these results suggest that EF1 becomes more conformationally labile at lower pH, perhaps because it is paired with EF4, which does not bind metal ions tightly.

For comparison of Trp-LanM performance with other luminescence-based Tb sensors, LODs and limits of quantification (LOQs) were calculated as described herein. LODs of the best performing sensor at low pH, T90W, are below 5 ppb over the pH 3-7 range, even at 1 μM T90W (Table 5). Even at pH 3, the LOD values are similar to (or better than) the lowest values reported for other complexes, which are often reported at pH 7. These results suggested that T90W-LanM might be robust enough to detect Tb even in challenging environmental samples.

TABLE 5
Limits of detection (LODs) and limits of quantification (LOQs) for
Tb, in ppb, of T41W and T90W LanM variants, at pH 3-7, using 1
or 10 μM protein. Each number denotes the mean ± S.D. (n = 3).

| Construct | pH 7 | pH 5 | pH 4 | pH 3 |
|---|---|---|---|---|
| | LOD (ppb) | | | |
| T41W-LanM | 1.52 ± 0.03 | 0.38 ± 0.02 | 10.6 ± 0.6 | 19 ± 3 |
| T90W-LanM | 2.4 ± 0.1 | 3.2 ± 0.2 | 1.30 ± 0.03 | 4.8 ± 0.3 |
| (1 μM) | | | | |
| T90W-LanM | ND^{a} | ND | 2.4 ± 0.1 | 0.78 ± 0.02 |
| (10 μM) | | | | |
| | LOQ (ppb) | | | |
| T41W-LanM | 5.1 ± 0.1 | 1.3 ± 0.1 | 35 ± 2 | 62 ± 10 |
| T90W-LanM | 7.9 ± 0.4 | 10.6 ± 0.5 | 4.3 ± 0.1 | 16 ± 1 |
| (1 μM) | | | | |
| T90W-LanM | ND | ND | 8.0 ± 0.3 | 2.6 ± 0.1 |
| (10 μM) | | | | |

^{a}ND: not determined

Application to quantification of terbium in acid mine drainage. In order to stringently challenge the affinity and selectivity of Trp-LanM and assess its potential for environmental monitoring, the performance of the most promising construct, T90W-LanM, was tested in acid mine drainage. AMD is effluent from active and abandoned mines, by which natural processes leads to release of metals, including REEs, from ores. AMD is often enriched in the rarer and more valuable heavy REEs due to the mechanism of natural acid-based extraction processes. The presence of existing infrastructure required to treat AIMD sites and mitigate their environmental impact prior to release of the AMD into natural waters has motivated investigation into feasibility of extracting REEs from these sources, in order to convert waste into revenue streams. An estimated 770 to 3400 tonnes of REEs are potentially accessible per year from AMD in Pennsylvania and West Virginia (USA) alone; for comparison, current domestic consumption of REEs is estimated at 13,000 tonnes per year. A field-deployable sensor for specific REEs such as Tb, or even a simpler laboratory procedure for analysis, could enable more rapid assessments of the value of new sites for development for REE extraction, as well as inexpensive in-line monitoring of extraction processes once implemented, compared to current ICP-MS or XFM elemental quantification methods.

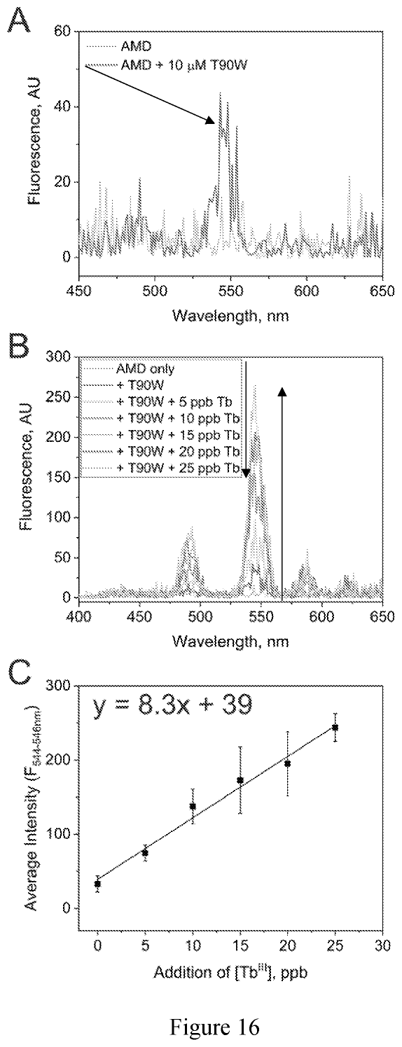

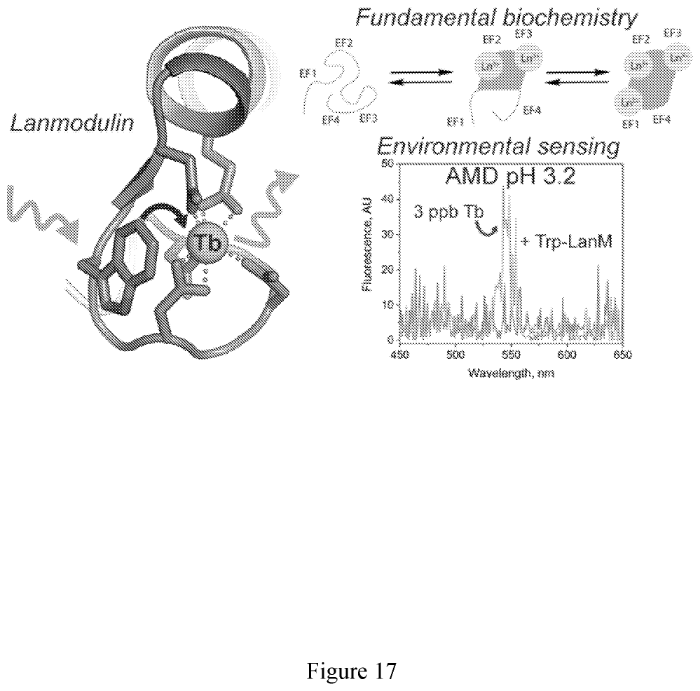

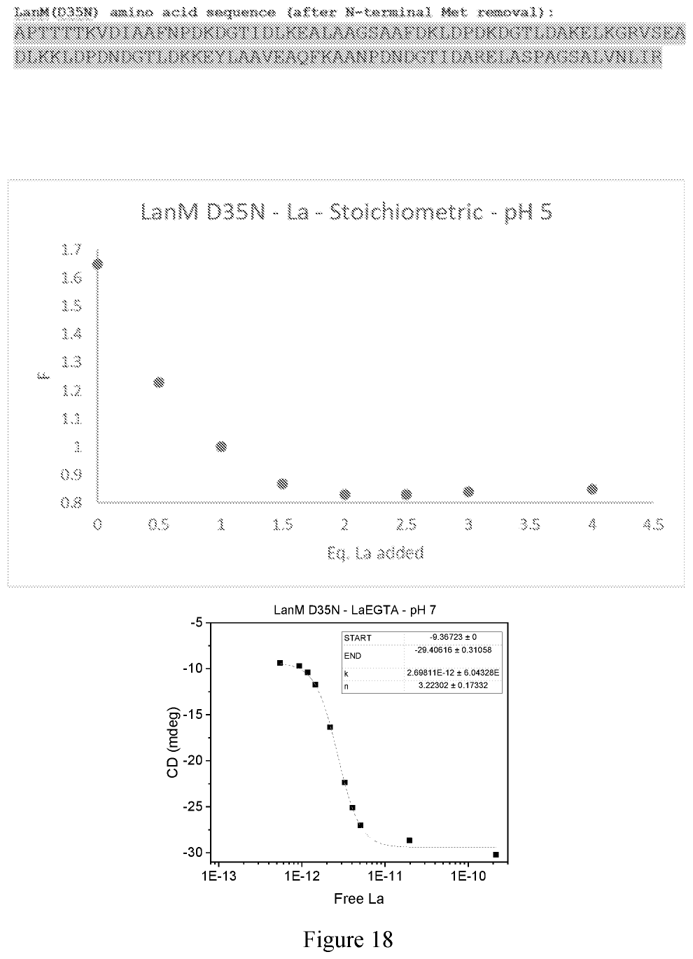

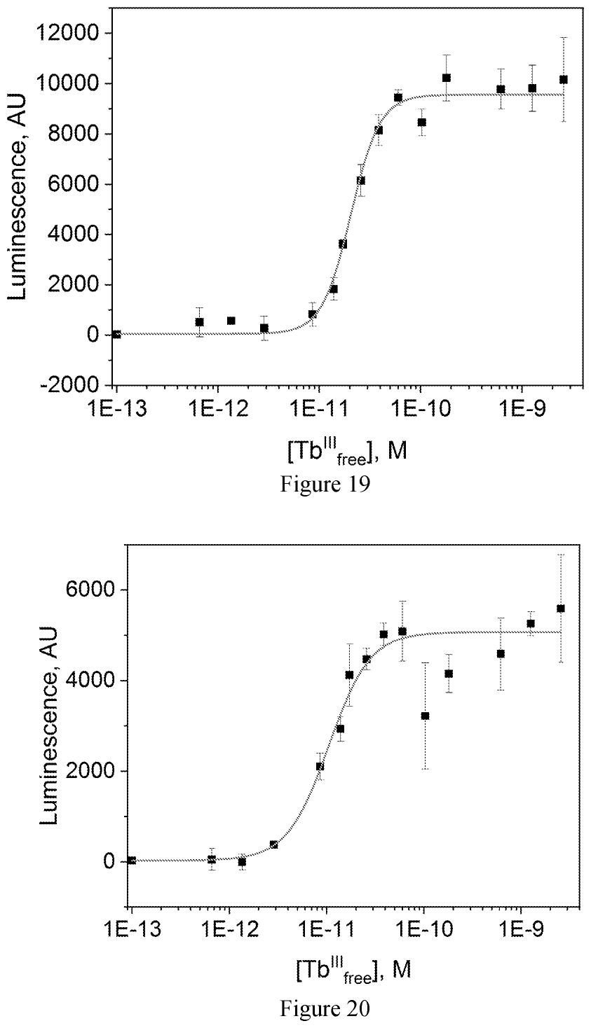

Samples of AMD (pH 3.24) collected from the feed of an AMD treatment facility in Pennsylvania, USA were used. Analysis of the sample using ICP-MS showed the presence of 300 ppm total metal ions, including high levels of potential interferents: 239 ppm (9.6 mM) Mg, 25 ppm (0.45 mM) Mn, 19 ppm (0.70 mM) Al, 12 ppm (0.41 mM) Si, and 1.5 ppm (22 μM) Zn (see Table 6 for the full analysis). The sample also contained 280 ppb total REEs and only 3.3 ppb Tb. As expected based on the LODs for T90W-LanM at pH 3 (Table 5), 1 μM sensor did not yield a luminescence signal clearly above background. However, when 10 μM T90W-LanM—a concentration far below the total metal concentration—was added, the Tb luminescence features at ˜490 and ˜545 nm were clearly visible above the background sample with no protein added (FIG. 16A). Whereas the combination of the 100-fold excess of REEs over Tb, 100,000-fold excess of non-REEs, and low pH would present a formidable challenge to most luminescence-based Tb detection methods, T90W-LanM's ability to detect Tb is consistent with LanM's high affinity for REEs and negligible binding of non-REEs, previously observed.

TABLE 6
ICP-MS analysis of the acid mine drainage sample used for
Tb detection, with elements listed in order of atomic
number. V, Cr, Zr, and Th were analyzed but not detected.

| Element | ppb | Concentration (M) |
|---|---|---|
| Li | 145.9 | 2.10 × 10⁻⁵ |
| Be | 11.98 | 1.33 × 10⁻⁶ |
| Na | 3994 | 1.74 × 10⁻⁴ |
| Mg | 233000 | 9.59 × 10⁻³ |
| Al | 18940 | 7.02 × 10⁻⁴ |
| Si | 11530 | 4.10 × 10⁻⁴ |
| K | 3217 | 8.23 × 10⁻⁵ |
| Ca | 3109 | 7.76 × 10⁻⁵ |
| Sc | 2.85 | 6.33 × 10⁻⁸ |
| Ti | 292.2 | 6.10 × 10⁻⁶ |
| Mn | 24790 | 4.51 × 10⁻⁴ |
| Fe | 424.2 | 7.60 × 10⁻⁶ |
| Co | 481.1 | 8.16 × 10⁻⁶ |
| Ni | 736.6 | 1.25 × 10⁻⁵ |
| Cu | 18.23 | 2.87 × 10⁻⁷ |
| Zn | 1454 | 2.22 × 10⁻⁵ |
| Sr | 53.01 | 6.05 × 10⁻⁷ |
| Y | 93.35 | 1.05 × 10⁻⁶ |
| Cd | 1.27 | 1.13 × 10⁻⁸ |
| Ba | 8.61 | 6.27 × 10⁻⁸ |
| La | 14.18 | 1.02 × 10⁻⁷ |
| Ce | 45.84 | 3.27 × 10⁻⁷ |
| Pr | 7.10 | 5.04 × 10⁻⁸ |
| Nd | 36.44 | 2.53 × 10⁻⁷ |
| Sm | 12.40 | 8.25 × 10⁻⁸ |
| Eu | 3.56 | 2.35 × 10⁻⁸ |
| Gd | 20.71 | 1.32 × 10⁻⁷ |
| Tb | 3.32 | 2.09 × 10⁻⁸ |
| Dy | 18.87 | 1.16 × 10⁻⁷ |
| Ho | 3.53 | 2.14 × 10⁻⁸ |
| Er | 9.31 | 5.56 × 10⁻⁸ |
| Tm | 1.08 | 6.42 × 10⁻⁹ |
| Yb | 6.68 | 3.86 × 10⁻⁸ |
| Lu | 0.88 | 5.01 × 10⁻⁹ |
| Pb | 1.37 | 6.63 × 10⁻⁹ |
| U | 1.48 | 6.22 × 10⁻⁹ |

Because components of the AMD matrix (other metal ions, as well as anions) could affect quantification, a standard curve was generated in the AMD itself by adding 0-25 ppb Tb^{III} in the presence of 10 μM T90W-LanM (FIG. 16B). Because the luminescence of the AMD in the absence of protein is negligible, dividing the intensity of the AMD sample without added Tb (33±11) by the slope of the regression line (8.3) yields a Tb concentration of 4.0±1.3 ppb (FIG. 16C). This value, close to but above the limit of quantification at pH 3, is in excellent agreement with the value determined by ICP-MS, 3.3 ppb. Because the slope of the line is only 50% lower than those of the standard curves obtained in idealized Tb solutions at pH 3 and pH 4, we suggest that the LODs determined may be limited more so by the sensitivity of the plate reader than by affinity or selectivity of the protein. Using more sensitive detectors (e.g., a spectrofluorometer), lower LODs could conceivably be achieved. Therefore T90W-LanM is an exceptionally sensitive sensor for Tb, applicable even in complex, environmental samples such as AMD.

The performance of Trp-LanM (and T90W-LanM specifically) in detecting Tb at low concentration even in complex media compares favorably with other luminescence-based sensors, both biomolecular and synthetic. Perhaps most conceptually similar to Trp-LanM is the LBT, but its affinity for Tb is only 60 nM at pH 7. Because this affinity is 3-4 orders of magnitude lower than that of LanM, LBTs would not be expected to function at pH 3. Indeed, attempts to use LBTs for Tb^{III} binding and sensing at pH values below ˜5-6 have been unsuccessful. A cell-based sensor incorporating an LBT into a bacterial two-component system responds to as little as ˜0.2 μM (30 ppb) Tb at neutral pH, but also responds significantly to other metals (e.g., Ca^{II} at 50 μM) at concentrations that would be present in environmental samples (e.g., our AMD sample contains 3.11 ppm, or 78 μM, Ca). Numerous synthetic luminescence-based sensors for lanthanides, including Tb, have been characterized, with a wide range of detection limits. Some of these sensors have been characterized in natural samples, although at higher pH values than AMD and spiked with Tb. A particularly extensively explored approach in recent years utilizes metal-organic frameworks (MOFs), exhibiting better tolerance to lower pHs than LBTs and other sensors but also quenching from interactions with other ions due to low selectivity. One of the most promising examples is a zinc-adeninate MOF for detection of several lanthanides (BioMOF-100), yielding an LOD for Tb of 90 ppb in neutral water. Very recently, others have characterized even more sensitive MOFs with LODs of 6 ppb Tb in neutral water. However, when this sensor was applied in an AMD sample containing ˜1 ppb Tb at pH 3.4, the sample had to be spiked with 800 ppb (5 μM) Tb to be able to observe a signal. By contrast, simply adding T90W-LanM directly to AMD detects and quantifies 3 ppb Tb present in AMD at pH 3.24, without any spiking. This comparison shows that the high affinity and selectivity of LanM for lanthanides provides a significant advantage over conventional REE sensitizer ligands, which (while they may have high affinity), usually do not have sufficient selectivity to work well in complex solutions like AMD.

### CONCLUSION

Incorporation of Trp residues site-specifically into LanM's metal-binding sites not only provides insights into the protein's metal recognition but also yields a technology capable of specific detection of terbium, even in complex samples. Established herein are several aspects of LanM function. First, these results indicate EF2 and EF3 as LanM's preferred metal-binding sites and responsible for the cooperative binding phase. EF1 is slightly lower affinity, but appears to be more destabilized at low pH values, even though metal binding is retained. This result suggests that EF1 might be the most dispensable site for applications of LanM to REE extraction and separations. In addition, it was shown that LanM-bound Tb^{III} ions possess two coordinated solvent molecules. The presence of these coordinated solvent molecules may explain the observation that LanM's association kinetics are extremely and uncommonly fast, perhaps minimizing the energetic penalty for dehydration upon metal binding. Rapid kinetics may be useful for separations applications, whereas these results also suggest that it may be possible to mutate the protein to decrease metal dissociation rates for other applications. Furthermore, these data establish EF3, and particularly the T90 position, as a location for installation of sensitizers within the protein scaffold with acceptable perturbation of metal binding and strong responsiveness across a wide pH range. Other lanthanides may be detected besides Tb via their unique luminescence signatures, including other valuable REEs such as Eu, Dy, Sm, and Nd, but Trp is unlikely to be a suitable sensitizer for these elements. Some of these sensitizers suitable could include, but are not limited to, unnatural Trp analogs (e.g., aza-tryptophans, including 4-aza, 5-aza, and 7-aza-tryptophans; cyano-tryptophans; and the boron- and nitrogen-containing BN-tryptophan) as well as naphthalimides, coumarins, acridones (e.g., acridon-2-ylalanine residues), and other fluorophores, which might be installed using auxotrophic strains, genetic code expansion methodologies, or via reaction with a Cys or other nucleophilic residue. Finally, the related coordination chemistry of the trivalent actinides suggests that Trp-LanMs may also serve as efficient sensitizers for some of these elements, such as Cm^{III} or perhaps even Am^{III}.

The information herein can be used to design screens to alter metal selectivity in LanM—for example, to increase differences in affinity between one REE and another REE (e.g., Nd vs. Dy, or Nd vs. Tb, or Tb vs. Dy, or any other combination of REEs) and thereby to increase affinity differences across the entire REE series. First, the present disclosure suggests that the most productive EF hands to re-engineer metal selectivity would be EF hands 2 and 3, and not EF1. EF1 could be disabled with minimal impact on the rest of the protein via the D35N substitution. Second, the characterization of optimal positions for luminescent sensitizers can be used in assay design. K94W is the least perturbative position, giving a luminescent probe of Tb binding to EF3; while the luminescence intensity of this variant is low, it may still be sufficient for a screening assay. It may be possible to, analogously, substitute the 11^{th} position of EF2 as well. N87W is slightly defective in Tb binding, and T90W is more so, but the LRET signal is strong and this could also be a suitable position for a screening assay. For example, one could start with the K94W variant, randomize various amino acids within EF2 and/or EF3 and/or in the vicinity of these EF hands (except for Trp94 itself), add TbCl₃ to saturate the protein binding sites, and then screen for most (or least) efficient outcompetition of the Trp-LanM-sensitized Tb luminescence signal by different concentrations of Nd, or Dy, or any other REE. Such a screening setup would provide an inherent comparison of the affinities of the protein for Tb versus any other REE in an effort to maximize those affinity differences to aid in REE separations. Similar experiments could be envisioned to alter metal selectivity from the REEs altogether, such as for uranyl (U^{VI}), or lithium (Li⁺), or manganese (Mn^{II}), or iron (Fe^{II}), or cobalt (Co^{II}), or any other element in its ionic form. Numerous variations of the above approach can be envisioned and the above description is not intended to be limiting.

The adaptability of this system to readily available luminescence detectors motivates further exploration and application of protein-based systems for rapid and inexpensive quantification of specific f-elements in natural sources and in separation processes.

### Example 2

This example provides a description of a protein of the present disclosure and methods of making and using same.

Methods. All protein expression and purification and LRET titrations were performed analogously to the previous description (Featherston et al., JACS2021).

Rationale. Initial experiments (in the provisional application) revealed that, in general, the seventh positions of LanM's EF hands were most suitable for tryptophan placement in EF hands 1, 3, and 4, but installing this mutation at this position in EF hand 2 (T65W) disrupted protein function. Stoichiometric Tb^{III} titrations monitored by CD showed that T65W displayed less helicity in the apoprotein as compared to WT LanM, as well as a nearly completely disrupted conformational response up to 5 equivalents of Tb^{III}. Nevertheless, these experiments did reveal the particular importance of EF2 in the metal-responsive conformational change of lanmodulin, making it desirable to be able to probe metal binding in this EF hand, as previously done. It was rationalized that Trp placement within EF2 further away from the EF2-EF3 interface would be less disruptive to metal binding and conformational change. The data in Example 1 showed that the 11^{th} position of EF3 (K94W) did not perturb metal binding, thus a Trp was tested at the 11^{th} position in EF2 (K69W). Because LRET efficiency was rather low in K94W, an alternative Trp substitution site in EF2, the 4^{th} position (K62W) was also tested.

TABLE 7
Amino acid sequences of the Trp-LanM variants used in these
studies. Mutation sites are indicated in bold.

| Construct | Sequence |
|---|---|
| T41W | APTTTTKVDIAAFDPDKDGWIDLKEALAAGSAAFDKLDPDKDGTL |
| | DAKELKGRVSEADLKKLDPDNDGTLDKKEYLAAVEAQFKAANPD |
| | NDGTIDARELASPAGSALVNLIR (SEQ ID NO: 11) |
| K62W | APTTTTKVDIAAFDPDKDGTIDLKEALAAGSAAFDKLDPDWDGTLD |
| | AKELKGRVSEADLKKLDPDNDGTLDKKEYLAAVEAQFKAANPDN |
| | DGTIDARELASPAGSALVNLIR (SEQ ID NO: 19) |
| T65W | APTTTTKVDIAAFDPDKDGTIDLKEALAAGSAAFDKLDPDKDGWL |
| | DAKELKGRVSEADLKKLDPDNDGTLDKKEYLAAVEAQFKAANPD |
| | NDGTIDARELASPAGSALVNLIR (SEQ ID NO: 12) |
| K69W | APTTTTKVDIAAFDPDKDGTIDLKEALAAGSAAFDKLDPDKDGTLD |
| | AWELKGRVSEADLKKLDPDNDGTLDKKEYLAAVEAQFKAANPDN |
| | DGTIDARELASPAGSALVNLIR (SEQ ID NO: 34) |
| T90W | APTTTTKVDIAAFDPDKDGTIDLKEALAAGSAAFDKLDPDKDGTLD |
| | AKELKGRVSEADLKKLDPDNDGWLDKKEYLAAVEAQFKAANPDN |
| | DGTIDARELASPAGSALVNLIR (SEQ ID NO: 14) |
| K94W | APTTTTKVDIAAFDPDKDGTIDLKEALAAGSAAFDKLDPDKDGTLD |
| | AKELKGRVSEADLKKLDPDNDGTLDKWEYLAAVEAQFKAANPDN |
| | DGTIDARELASPAGSALVNLIR (SEQ ID NO: 15) |
| T114W | APTTTTKVDIAAFDPDKDGTIDLKEALAAGSAAFDKLDPDKDGTLD |
| | AKELKGRVSEADLKKLDPDNDGTLDKKEYLAAVEAQFKAANPDN |
| | DGWIDARELASPAGSALVNLIR (SEQ ID NO: 16) |

Results. Both K62W and K69W resulted in efficient luminescence as measured by LRET. Both constructs have K_{d,app} and Hill coefficients relatively close to the CD-derived values for WT-LanM. The overall change in magnitude in luminescence is greater for K62W then K69W and shows greater reproducibility. The maximum luminescence of K62W is ˜0 that of T90W. Therefore, K62W is the best position of those tested for Trp insertion to EF2 in terms of LRET data.

TABLE 8
K_{d, app} and Hill coefficients (n) for K62W- and K69W-LanM.

| Construct | K_{d, app} (pM) | n |
|---|---|---|
| K62W-LanM | 21 ± 2 | 3.0 ± 0.8 |
| K69W-LanM | 10.5 ± 0.9 | 2.0 ± 0.2 |

Although the present disclosure has been described with respect to one or more particular embodiments and/or examples, it will be understood that other embodiments and/or examples of the present disclosure may be made without departing from the scope of the present disclosure.

1. A protein having the sequence or comprising a segment having the sequence of SEQ ID NO:2 or sequence having at least 80% homology thereto, wherein at least one amino acid residue of the protein or segment is replaced with a sensitizer or modified such that the sensitizer is attached to the protein or the segment.

2. The protein according to claim 1, wherein the sensitizer is chosen from tryptophan, tryptophan analogs, naphthalimides, coumarins, acridones, and combinations thereof.

3. The protein according to claim 2, wherein the protein is any one of sequences SEQ ID NO:43-50 or a sequence having 80% homology thereto to any one of sequences SEQ ID NO:43-50.

4. The protein according to claim 2, wherein the protein is any one of sequences SEQ ID NO:43-50.

5. The protein according to claim 2, wherein the sensitizer is tryptophan.

6. The protein according to claim 5, wherein the protein is any one of sequences SEQ ID NO:11-19 or 34 or a sequence having 80% homology thereto to any one of sequences SEQ ID NO: 11-19 or 34.

7. The protein according to claim 6, wherein the protein is any one of sequences SEQ ID NO:11-19 or 34.

8. The protein according to claim 7, wherein the protein has the sequence SEQ ID NO:19 or SEQ ID NO:14.

9. A device comprising the protein of claim 1.

10. The device according to claim 9, wherein the device is a filter, membrane, sensor, handheld detector, plate reader, fluorimeter, biosensor, or in-line monitor.

11. A kit comprising the protein according to claim 1 or a device comprising the protein according to claim 1.

12. A method for binding one or more lanthanides and/or actinides comprising contacting a sample suspected of comprising or comprising the one or more lanthanides and/or actinides with one or more proteins according to claim 1, wherein one or more lanthanides and/or actinides binds to the protein.

13. The method according to claim 12, wherein the sample is drinking water, wastewater, ground water, ash ponds, aqueous extract from contaminated soil, drainage, leachate, or solid sample.

14. The method according to claim 12, wherein the one or more lanthanides are chosen from Tb, Eu, Dy, Sm, Nd, and ions thereof.

15. The method according to claim 14, wherein the lanthanide is Tb or an ion thereof.

16. The method according to claim 12, wherein the one or more actinides are americium, curium, or an ion thereof.

17. The method according to claim 12, wherein the sample has a pH of 9 or below.

18. The method according to claim 12, wherein the concentration of the one or more lanthanides and/or actinides is less than 100 ppm.

19. The method according to claim 12, wherein the bound one or more lanthanides and/or actinides are unbound and isolated from the protein.

20. A method for detecting and quantifying one or more lanthanides and/or actinides in a sample, comprising contacting the sample with one or more proteins according to claim 1;
exposing the contacted sample with light;
measuring a resulting emission of the exposed contacted sample;
comparing the resulting emission to a known standard curve for a specific lanthanide; and
determining the concentration of the specific lanthanide or actinide based on the comparison with the resulting emission and the known standard curve for the specific lanthanide or actinide,
wherein the one or more lanthanides and/or actinides, if present, are detected and quantified based on the comparison of the resulting emission and the known standard curve.

21. The method according to claim 20, wherein the sample is drinking water, wastewater, ground water, ash ponds, aqueous extract from contaminated soil, drainage, leachate, or solid sample.

22. The method according to claim 20, wherein the one or more lanthanides are chosen from Tb, Eu, Dy, Sm, Nd, and ions thereof.

23. The method according to claim 22, wherein the lanthanide is Tb or an ion thereof.

24. The method according to claim 20, wherein the one or more actinides is americium, curium, or a combination thereof.

25. A method according to claim 20, wherein the sample has a pH of 9 or below.

26. A method according to claim 20, wherein the concentration of the one or more lanthanides and/or actinides is less than 100 ppm.
